(19) United States
(12) Patent Application Publication
(10) Pub. No.: US 2020/0176105 A1
(43) Pub. Date: Jun. 4, 2020

(54) DEVICES AND METHODS FOR GUIDING AND RECORDING EMERGENCY MEDICAL TREATMENT

(71) Applicants: Brian Ross, Seattle, WA (US); Bala G. Nair, Seattle, WA (US); Roland Lai, Seattle, WA (US); Peter E. Oppenheimer, Seattle, WA (US); Timothy Wu, Seattle, WA (US); Brian Howe, Seattle, WA (US)

(72) Inventors: Brian Ross, Seattle, WA (US); Bala G. Nair, Seattle, WA (US); Roland Lai, Seattle, WA (US); Peter E. Oppenheimer, Seattle, WA (US); Timothy Wu, Seattle, WA (US); Brian Howe, Seattle, WA (US)

(73) Assignee: University of Washington, Seattle, WA (US)

(21) Appl. No.: 16/206,846

(22) Filed: Nov. 30, 2018

(51) Int. Cl. G16H 40/20; G16H 70/20; G06Q 10/06; G06F 3/0488; G06F 3/0485

(52) U.S. Cl. CPC G16H 40/20; G16H 70/20; G06F 3/0485; G06F 3/04883; G06Q 10/06316

(57) ABSTRACT

Embodiments of the present disclosure include devices, systems, and methods for managing an emergency care event using a computing device. Simplified and touch-enabled user interface elements allow a wide variety of events to be selected and quickly entered into an event record. In some embodiments, care guidelines are programmed into the computing device, and are used by the computing device to guide the care team through the steps for various actions to ensure that adherence to the care guidelines is as close as possible. In some embodiments, timers for care events are automatically tracked by the computing device, and alerts are automatically generated in order to further increase compliance with time-based care guidelines.

### STATEMENT OF GOVERNMENT LICENSE RIGHTS

This invention was made with Government support under W81XWH-10-2-0023 awarded by the Department of Defense. The Government has certain rights in the invention.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a mobile computing device for managing and logging actions during an emergency care event is provided. The mobile computing device comprises a touch-enabled display device, one or more processors, an event data store, a communication interface, a non-transitory computer-readable medium. The non-transitory computer-readable medium has computer executable instructions stored therein that, in response to execution by the one or more processors, cause the computing device to present a first modal interface element to collect information that defines at least one care guideline, and close the modal interface element after collecting the information; in response to detecting actuation of an event-initiating interface element, present a non-modal interface to manage and record event information, wherein content of the at non-modal interface element is determined by the at least one care guideline; and store collected information in an event record in the event data store.

In some embodiments, a non-transitory computer-readable medium having computer-executable instructions stored thereon is provided. The instructions, in response to execution by one or more processors of a mobile computing device, cause the mobile computing device to perform actions for managing and logging actions during an emergency care event, the actions comprising: presenting a first modal interface element to collect information that defines at least one care guideline, and close the modal interface element after collecting the information; in response to detecting actuation of an event-initiating interface element, presenting a non-modal interface to manage and record event information, wherein content of the at non-modal interface element is determined by the at least one care guideline; and storing collected information in an event record in the event data store.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram that illustrates an example embodiment of an emergency care management device according to various aspects of the present disclosure;

FIGS. 2A-2C are a flowchart that illustrates an example embodiment of a method of managing and logging actions during an emergency care event according to various aspects of the present disclosure;

FIG. 3 is a flowchart that illustrates an example embodiment of a procedure for managing and recording a treatment cycle item according to various aspects of the present disclosure;

FIG. 4 is a flowchart that illustrates an example embodiment of a procedure for managing and recording a workflow item according to various aspects of the present disclosure;

FIGS. 5A-5B are a flowchart that illustrates an example embodiment of a procedure for managing and recording an order item according to various aspects of the present disclosure;

FIG. 6 is a block diagram that illustrates aspects of a representative computing device appropriate for use with embodiments of the present disclosure; and

FIGS. 7-38 illustrate non-limiting example embodiments of interfaces presented by an emergency care management device according to various aspects of the present disclosure.

### DETAILED DESCRIPTION

There are approximately 200,000 in-hospital cardiac arrests annually in the United States, and—being chaotic and transitory—these events are difficult to manage and document accurately. Thorough documentation is essential for real-time patient care, as well as for handoff and subsequent care, institutional quality improvement, research investigations, and medico-legal reviews. While electronic records have replaced paper records in many facets of healthcare, their ability to document emergent situations is unproven, particularly since entering information into existing electronic systems is often cumbersome and inefficient.

Due to these inefficiencies, documentation of medical emergencies, where many different actions to be recorded happen in a short amount of time, continues to be performed on paper records. Unfortunately, paper records of medical emergency care are generally inaccurate and incomplete because they are visually dense and structurally complex, are typically filled out by personnel unfamiliar with the form, and are typically completed after an event. In a review of paper records at the University of Washington, it was found that information such as “reason resuscitation ended” was only recorded 45% of the time.

In addition to these difficulties in recording emergency care events, guiding emergency care is also a difficult task. Though care guidelines exist that can help improve outcomes, adhering to these guidelines can be difficult in the chaotic setting of an emergency response. One example of such a guideline is the Advanced Cardiac Life Support (ACLS) guideline published by the American Heart Association. According to a study by M. D. McEvoy et al., “The effect of adherence to ACLS protocols on survival of event in the setting of in-hospital cardiac arrest,” 85 Resuscitation 82-87 (2013), adherence to the ACLS protocols throughout an emergency care event is associated with increased return of spontaneous circulation (ROSC) in an in-hospital cardiac arrest, and that wrong actions or omitted actions from the ACLS protocols are associated with decreased ROSC. The study also showed poor adherence to the ACLS protocols, despite the fact that adherence could be shown to be associated with improved outcomes, and that quality of CPR was an important factor.

Since both care guidance and event recordation can be provided by a computing device, the complications that prevent the use of a computing device for such tasks are best construed a technical problem. In order to solve these technical problems, and to therefore improve patient care and outcomes, embodiments of the present disclosure include devices, systems, and methods for managing an emergency care event using a computing device. Simplified and touch-enabled user interface elements allow a wide variety of events to be selected and quickly entered into an event record. In some embodiments, care guidelines are programmed into the computing device, and are used by the computing device to guide the care team through the steps for various actions to ensure that adherence to the care guidelines is as close as possible. In some embodiments, timers for care events are automatically tracked by the computing device, and alerts are automatically generated in order to further increase compliance with time-based care guidelines.

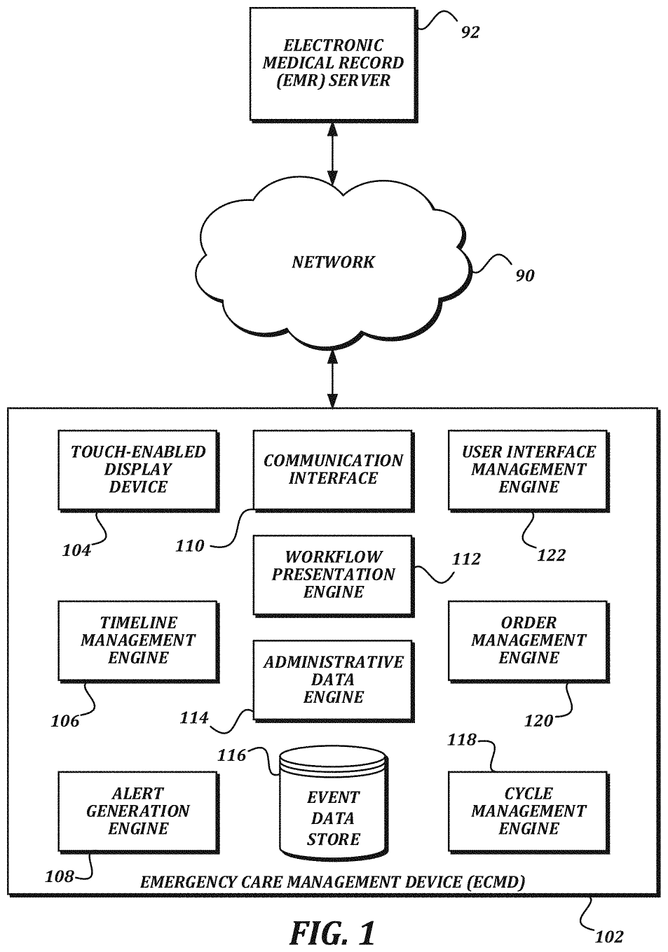

FIG. 1 is a block diagram that illustrates an example embodiment of an emergency care management device according to various aspects of the present disclosure. In some embodiments, the emergency care management device (ECMD) 102 is a tablet computing device. Some non-limiting examples of tablet computing devices include an Apple iPad, a Samsung Galaxy Note, an Amazon Kindle Fire, and a Microsoft Surface. Using a tablet computing device has technical benefits, in that a tablet computing device is highly portable and can be operated in a handheld manner during an emergency care event without requiring any additional input devices such as a keyboard or mouse.

As illustrated, the ECMD 102 communicates with an electronic medical record (EMR) server via a network 90. In some embodiments, the network 90 (or portions thereof) may include one or more wireless networks that use any available wireless communication technology, including but not limited to Wi-Fi, WiMAX, 2G, 3G, 4G, LTE, and Bluetooth. In some embodiments, the network 90 (or portions thereof) may include one or more wired networks that use any available wired communication technology, including but not limited to Ethernet, USB, FireWire, and the Internet.

In some embodiments, the EMR server 92 includes one or more computing devices configured to store health care related information, such as patient identity, vital signs, symptoms, treatments, medications, and demographic information. In some embodiments, instead of communicating directly with the EMR server 92, the ECMD 102 may instead communicate with an intermediate staging server, which receives event records from the ECMD via the network 90 (or a direct wired connection) and thereafter transmits the event records to the EMR server 92.

As shown, the ECMD 102 includes a touch-enabled display device 104 and a communication interface 110. The touch-enabled display device 104 displays an interface generated by the ECMD 102, registers one or more locations being touched by the user, and provides the touched locations to other components of the ECMD 102 as input data. In some embodiments, the touch-enabled display device 104 may register not only a location touched, but also an amount of force used. In some embodiments, the communication interface 110 may include one or more wireless network interfaces configured to communicate via the network 90 as described above. In some embodiments, the communication interface 110 may include one or more wired interfaces configured to either communicate with via the network 90, or to communicate directly with another computing device that will relay information to the EMR server 92 via the network 90. In some embodiments, the ECMD 102 may be configured to wirelessly mirror interfaces presented on the touch-enabled display device 104 (or transmit other interfaces) to another display device, such as a large-format monitor or projection screen, so that the entire care team can glance at the large-format display in order to view the timers, alerts, timelines, and other interface elements without having to ask the recording user for status.

The illustrated ECMD 102 also includes a number of engines, including a user interface management engine 122, a workflow presentation engine 112, a timeline management engine 106, an administrative data engine 114, an order management engine 120, an alert generation engine 108, and a cycle management engine 118.

In general, the word “engine,” as used herein, refers to logic embodied in hardware or software instructions, which can be written in a programming language, such as C, C++, COBOL, JAVA™, PHP, Perl, HTML, CSS, JavaScript, VBScript, ASPX, Microsoft .NET™, Swift, Go, and/or the like. An engine may be compiled into executable programs or written in interpreted programming languages. Software engines may be callable from other engines or from themselves. Generally, the engines described herein refer to logical engines that can be merged with other engines, or can be divided into multiple engines. The engines can be stored in any type of computer-readable medium or computer storage device and be stored on and executed by one or more processors of the ECMD 102, thus creating a special purpose computer configured to provide the engine. Thus, the term “engine” as used herein may be shorthand for one or more processors and a computer-readable medium having computer-executable instructions stored thereon that, in response to execution by the one or more processors, cause the one or more processors to perform the actions described as being performed by the engine. In some embodiments, communication between engines of the ECMD 102 may occur using any suitable technique, including but not limited to a transfer on a communication bus of the ECMD 102, a copy of a value from one location on a computer-readable medium to another location on the computer readable medium, or via a shared location on a computer-readable medium.

In some embodiments, the user interface management engine 122 is configured to generate an overall user interface for guiding care events and recording information. In some embodiments, the workflow presentation engine 112 is configured to provide functionality for guiding and recording an action that includes a sequence of steps that should each be performed in a specified order. In some embodiments, the timeline management engine 106 is configured to receive information regarding events, and to display and manage a timeline view of the event information. In some embodiments, the administrative data engine 114 is configured to record and manage administrative information describing the emergency care event that does not reflect a specific treatment provided. In some embodiments, the order management engine 120 is configured to record orders for medications, treatments, and other items, as well as tracking when/if the ordered items are actually administered. In some embodiments, the order management engine 120 is also configured to track ordered items that have not yet been administered in order to help ensure that they are not forgotten. In some embodiments, the alert generation engine 108 is configured to receive information from other components of the ECMD 102 for generating visual, audible, haptic, or other alerts, and to generate such alerts when appropriate. In some embodiments, the cycle management engine 118 is configured to track treatments that, according to guidelines, are performed in a timed cycle, and to present information to assist compliance with the cycle timing guidelines. Further description of the actions performed by each of these engines is provided below.

The engines of the ECMD 102 are illustrated and described herein as separate engines for ease of discussion, and to indicate categories of functionality provided by the ECMD 102. In some embodiments, functionality described as being performed by multiple engines may instead be performed by a single engine, or by one or more processors of the ECMD 102 in a non-differentiated manner. Accordingly, the description of functionality as being performed by any given engine should be seen as an example only, and should not be construed as limiting.

In some embodiments, the ECMD 102 also includes an event data store 116. A “data store” as described herein may be any suitable device configured to store data for access by a computing device. One example of a data store includes any data stored in an organized manner, such as files in a file system or data in a database, on a computer-readable storage medium, as described further below. In some embodiments, the event data store 116 may advantageously be stored on a computer-readable medium of the ECMD 102, such that a network connection to a server need not be established to operate the ECMD 102. However, any other suitable storage technique and/or device capable of quickly and reliably providing storage for data may be used, and the computing device may be accessible over a network instead of being incorporated into the ECMD 102. In some embodiments, the event data store 116 is configured to store information for one or more emergency care events. The information for a given emergency care event may be stored in an event record in the event data store 116. Further description of the information stored in the event record is provided below.

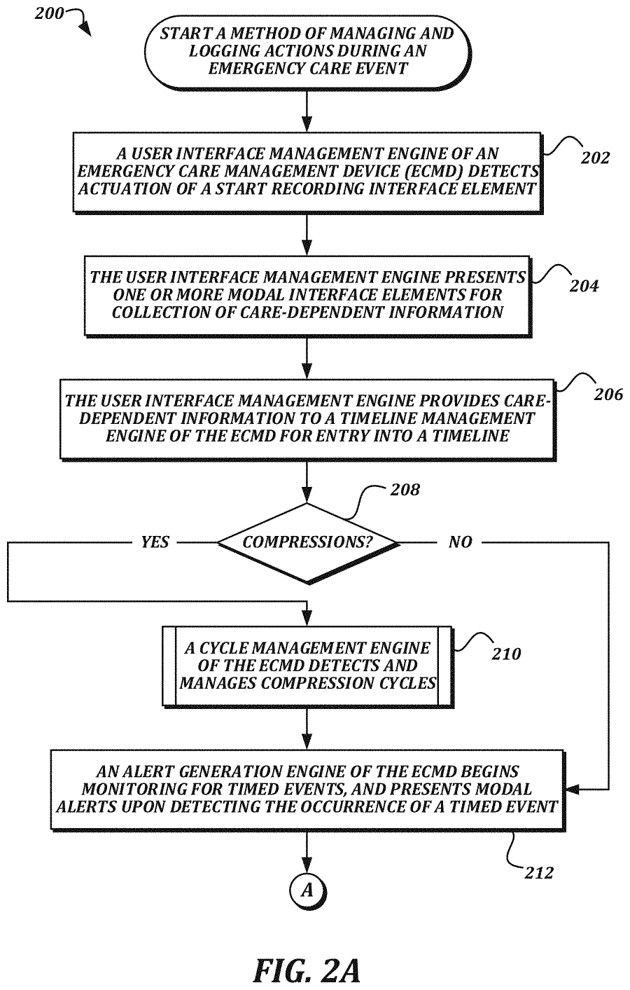

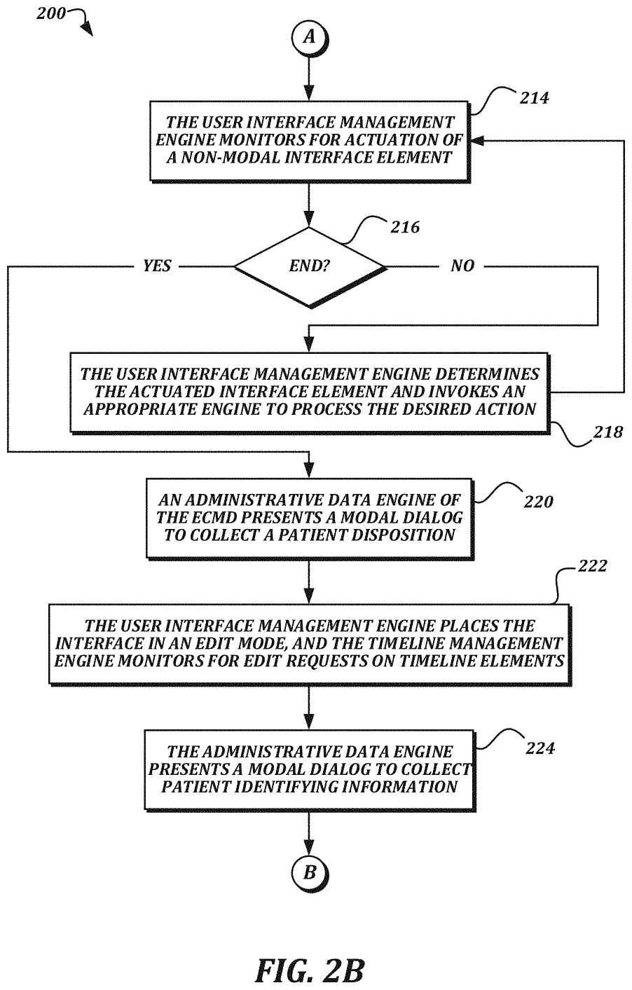

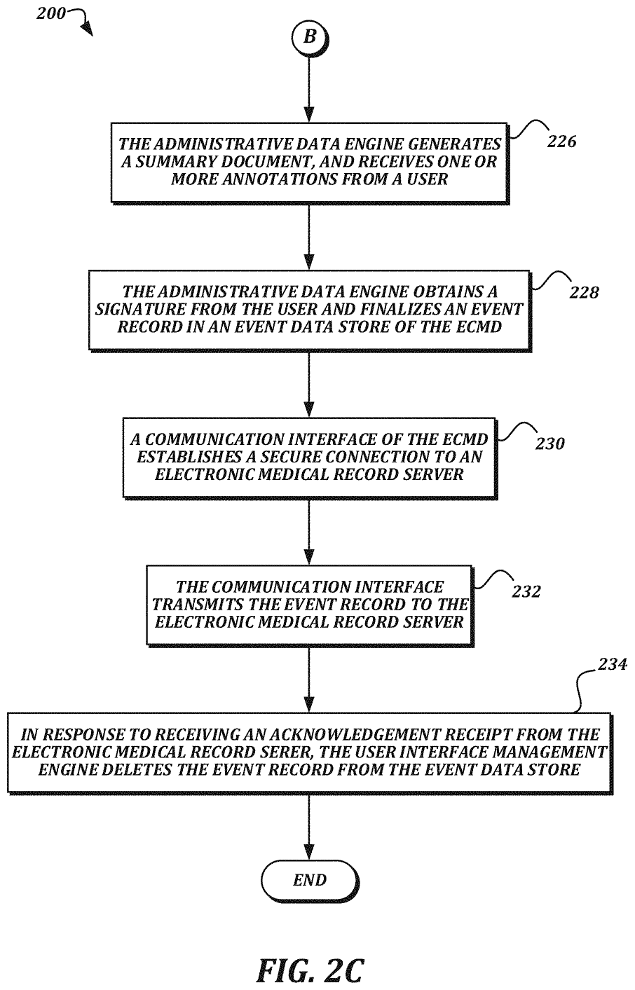

FIGS. 2A-2C are a flowchart that illustrates an example embodiment of a method of managing and logging actions during an emergency care event according to various aspects of the present disclosure. Typically, an emergency care event, such as a “code blue” to respond to a cardiac arrest, begins with a care team being paged and beginning treatment as soon as possible. Upon being paged, a recording user may retrieve the ECMD 102, which, due to the small and portable form factor, may be stored in the patient room, on a crash cart, or in any other suitable location. The recording user then uses the ECMD 102 to record actions that occur during the care event. For some actions, the ECMD 102 provides guidance to the recording user regarding steps that should be performed for those actions in order to comply with care guidelines, and the recording user can advise the rest of the care team regarding the steps. The ECMD 102 may also provide alerts when guidelines are not being followed, or to remind users when time-based events should occur. Once the care event has ended and any desired edits are made to the stored information, the ECMD 102 stores a signed, tamper-proof copy of the event record, and transmits the event record to the EMR server 92 for storage.

Many of the steps in the flowchart illustrated in FIGS. 2A-2C are shown as occurring in a sequential order, but this is only for ease of discussion and should not be seen as limiting. In some embodiments, many of the steps may actually occur at multiple times within the method 200, and/or concurrently with other steps of the method 200.

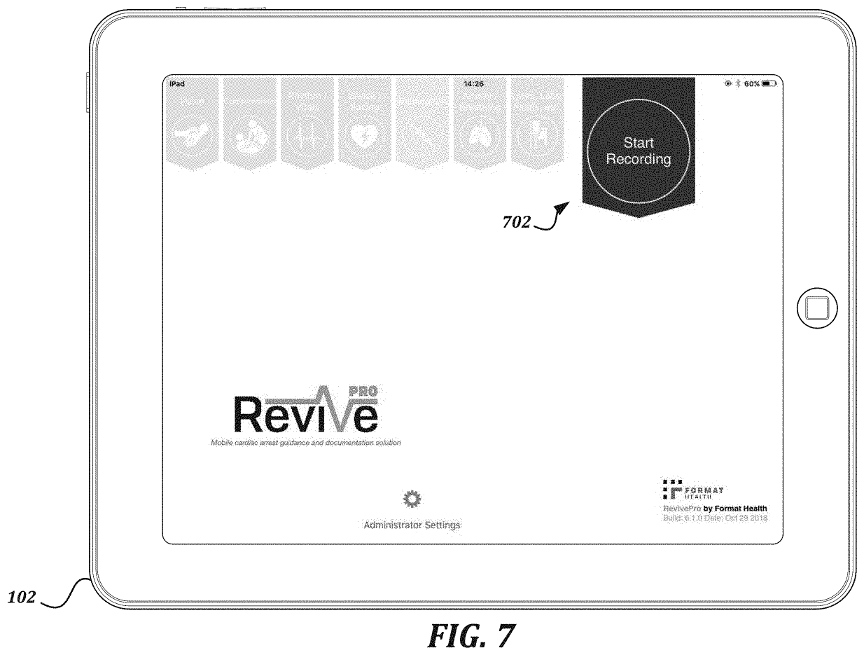

From a start block, the method 200 proceeds to block 202, where a user interface management engine 122 of an emergency care management device (ECMD) 102 detects actuation of a start recording interface element. Throughout this description, actuation of an interface element may include any way to actuate an interface element on a touch-enabled display device 104, including but not limited to tapping, tapping and holding, swiping, double-tapping, or hard-pressing. FIG. 7 illustrates an example embodiment of a start recording interface element 702 being presented by an ECMD 102 on a splash screen of an application executed by the ECMD 102.

At block 204, the user interface management engine 122 presents one or more modal interface elements for collection of care-dependent information. A modal interface element is a dialog window, form, or other interface element that is presented by the ECMD 102, and that must be dismissed, acknowledged, or otherwise interacted with before any other action can be taken within the interface. In some embodiments, when a modal interface element is displayed, other interface elements may be grayed out, dimmed, or otherwise deactivated in order to force input into only the modal interface element. In some embodiments, the care-dependent information is basic information that can be used as a basis for the ECMD 102 to guide care. Care-dependent information may include, but is not limited to, an age or age group (such as adult or pediatric), a weight, a medication status, and a current treatment being received. As non-limiting examples of guidelines that may be affected by care-dependent information, the interface may specify different steps for treating pediatric patients versus treating adult patients, and the interface may specify different recommended amounts of medications based on patient weight.

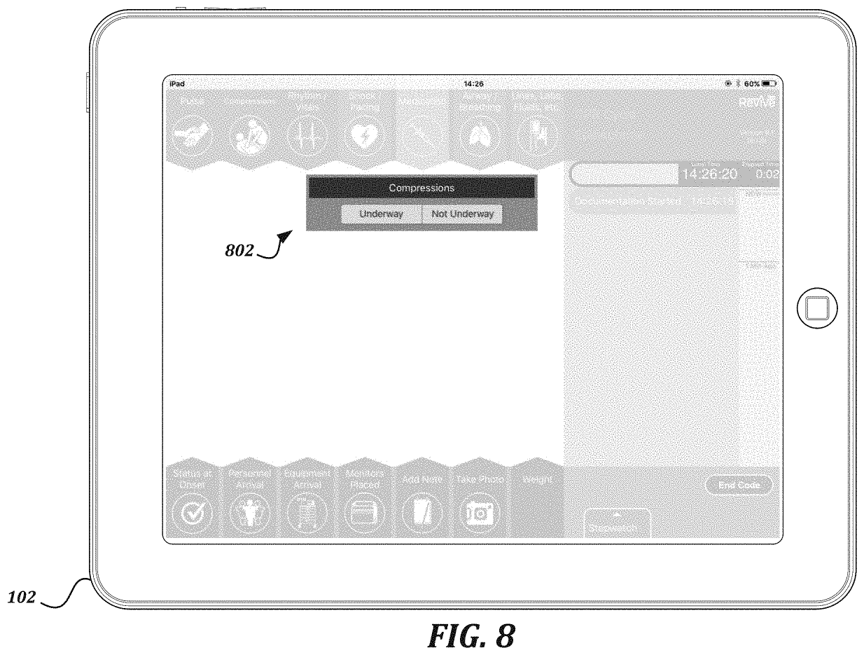

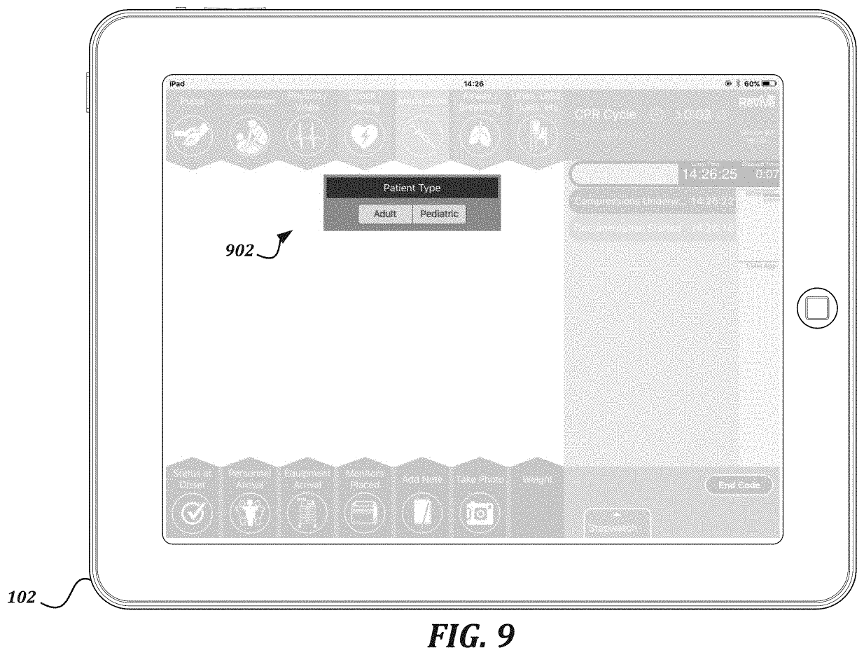

FIG. 8 illustrates a non-limiting example embodiment of an interface presented by the ECMD 102 that is presenting a modal interface element 802 to collect status/treatment information regarding whether chest compressions are underway. FIG. 9 illustrates a non-limiting example embodiment of an interface presented by the ECMD 102 that is presenting a modal interface element 902 to collect age information regarding whether the patient is an adult patient or a pediatric patient. In the embodiments illustrated in FIGS. 8 and 9, all elements other than the modal interface element have been disabled in order to force a selection in the modal interface element.

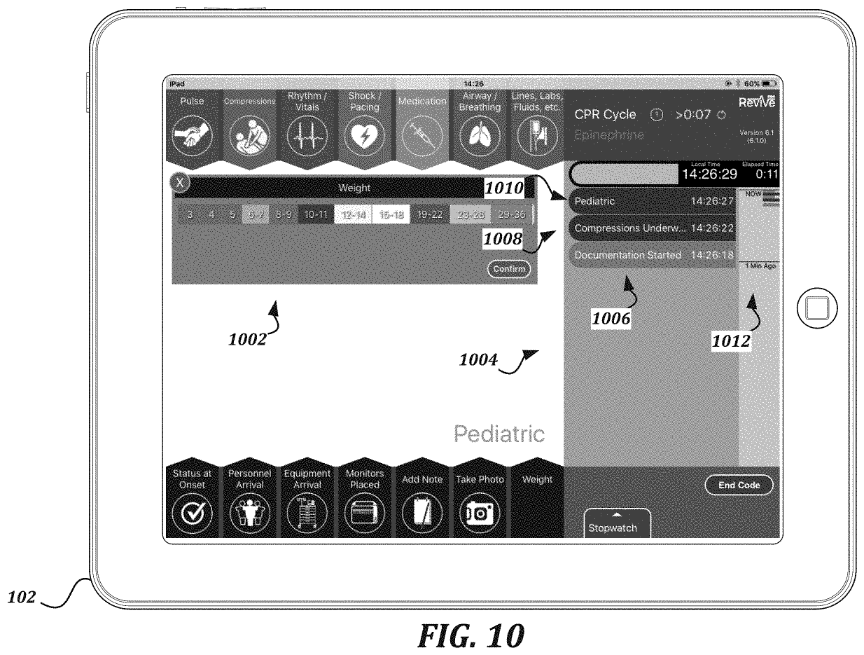

In some embodiments, care-dependent information may be collected in a non-modal interface element. In such embodiments, collection of the care-dependent information may be facilitated by automatic display of the non-modal interface element (rather than requiring a user to select an item to enter the information. FIG. 10 illustrates a non-limiting example embodiment of an interface presented by the ECMD 102 that collects weight information using a non-modal interface element 1002. The non-modal interface element 1002 includes a color coded list of weights for a pediatric patient, indicating that such an interface element 1002 may be presented in response to a selection made in the modal interface element 902 illustrated previously. Other examples of a non-modal interface element 1002 for collecting weight information may use other colors; or allow entry of other weights, such as for adults; or may be able to scroll by swiping on the interface to show weights other than those shown in FIG. 10. Other examples of care-dependent information may include other vital signs such as heart rate, temperature, respiration rate, oxygen saturation levels, and other information, though in the present example method 200, it is assumed that the ECMD 102 is managing a response to an acute event such as a cardiac arrest or respiratory arrest, and so detailed vital sign information may not be collected at the initiation of management of the event.

At block 206, the user interface management engine 122 provides care-dependent information to a timeline management engine 106 of the ECMD 102 for entry into a timeline. A timeline 1004 is shown in FIG. 10. Each entry of information is used by the timeline management engine 106 to create a timeline event, such as a first timeline event 1006 for the start of the recording, a second timeline event 1008 for the first entry of care-dependent information, and a third timeline event 1010 for the second entry of care-dependent information. Each of the timeline events includes a timestamp and a description of the event. In some embodiments, the timeline 1004 may be scrollable by dragging the list of timeline events. As will be shown later, tapping on a timeline event 1006, 1008, 1010 in the timeline 1004 may cause detailed information associated with the timeline event to be presented and/or edited. New timeline events may be added to the top of the timeline 1004, with all other timeline events being pushed down. A timeline overview 1012 may also be displayed, which shows discrete events, as well as ongoing events, in a condensed form to give an overview of when various events happened. In some embodiments, the timeline overview 1012 may auto-scroll from the top to the bottom in order to indicate the passage of time.

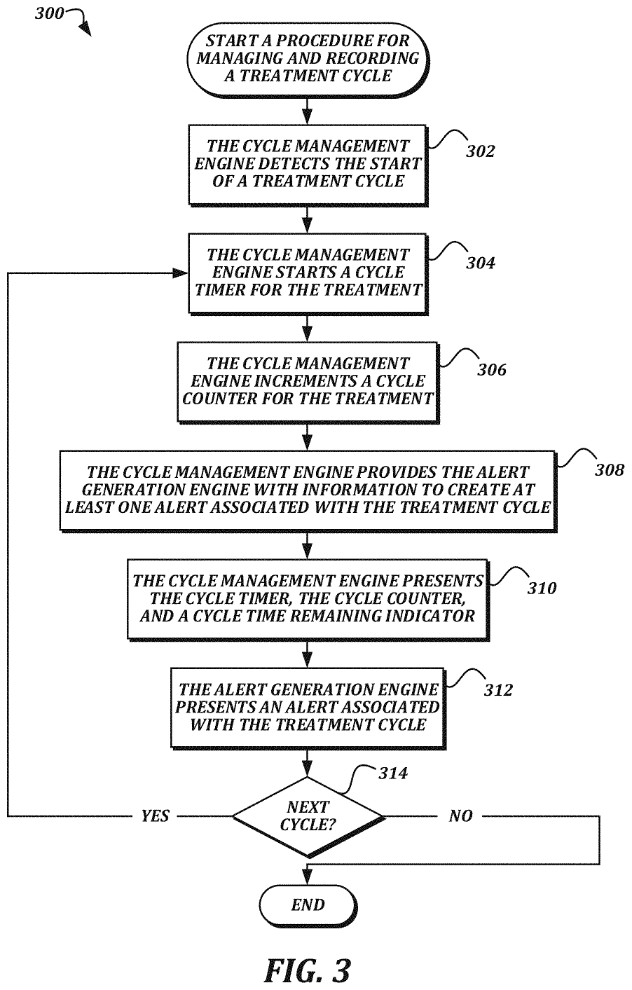

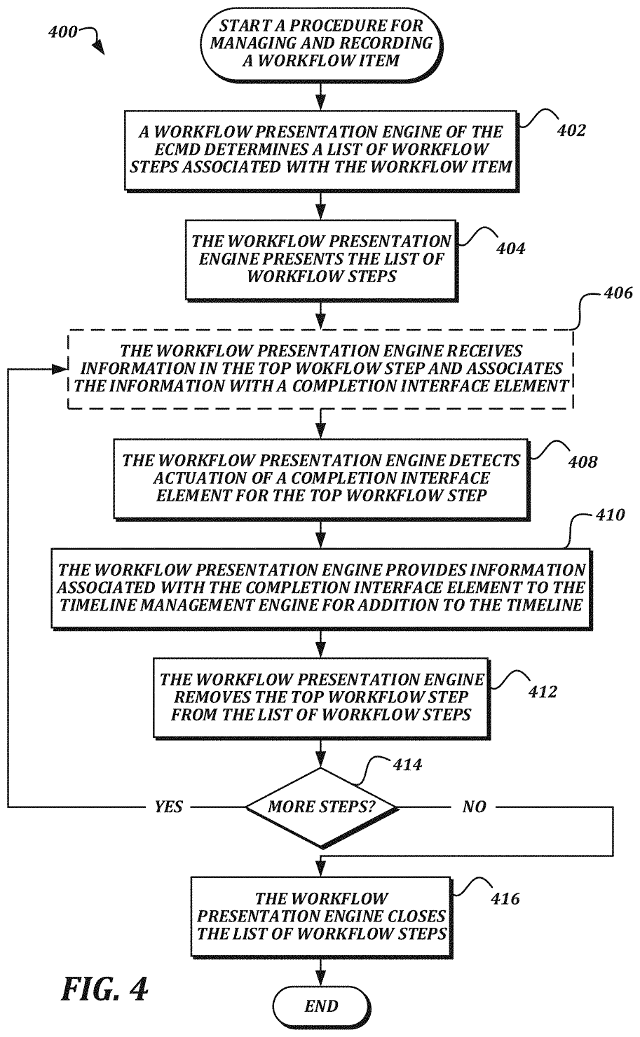

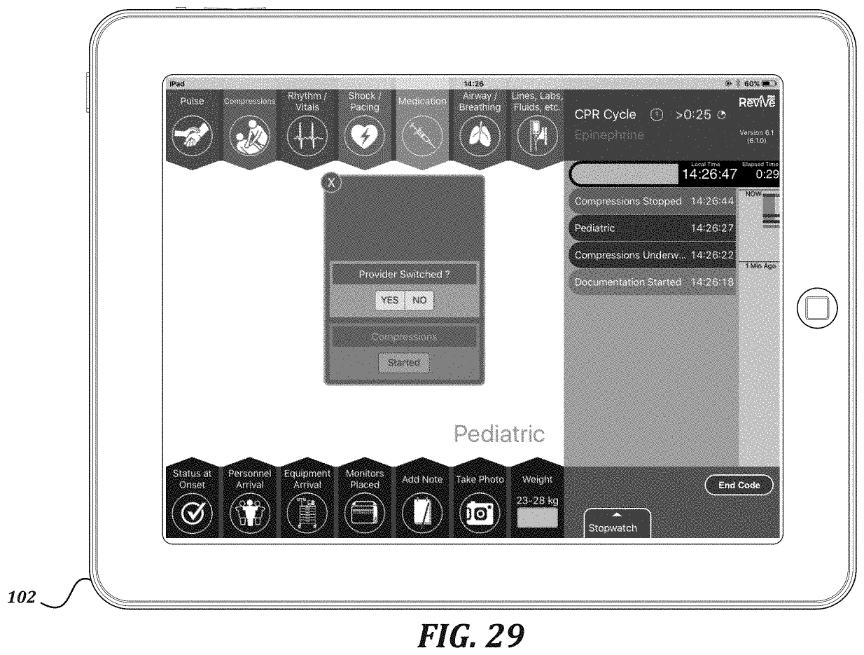

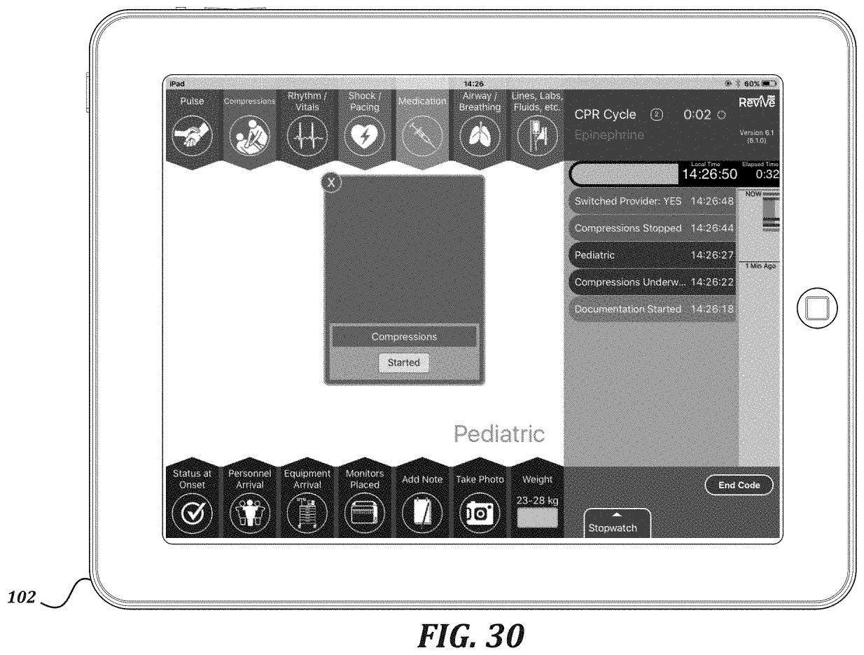

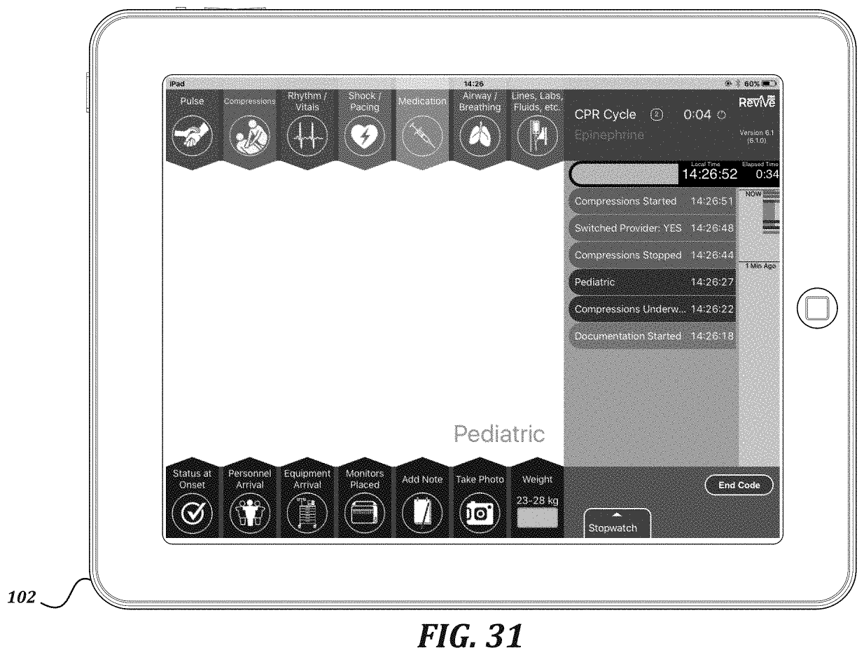

The method 200 then proceeds to a decision block 208, where a determination is made regarding whether chest compressions are being performed. In some embodiments, this determination may use a status indicated at block 204, as shown in FIG. 8. If it is determined that chest compressions are being performed, then the result of decision block 208 is YES, and the method 200 advances to a procedure block 210, where a procedure is executed wherein a cycle management engine 118 of the ECMD 102 detects and manages compression cycles. In some embodiments, the procedure may be executed in parallel with other steps of the method 200. In some embodiments, the workflow item management procedure 400 described further below and illustrated in FIGS. 4 and 28-31 may be used to guide changes between compression cycles, while the treatment cycle management procedure 300 illustrated in FIG. 3 and described further below may be used to count and time each compression cycle.

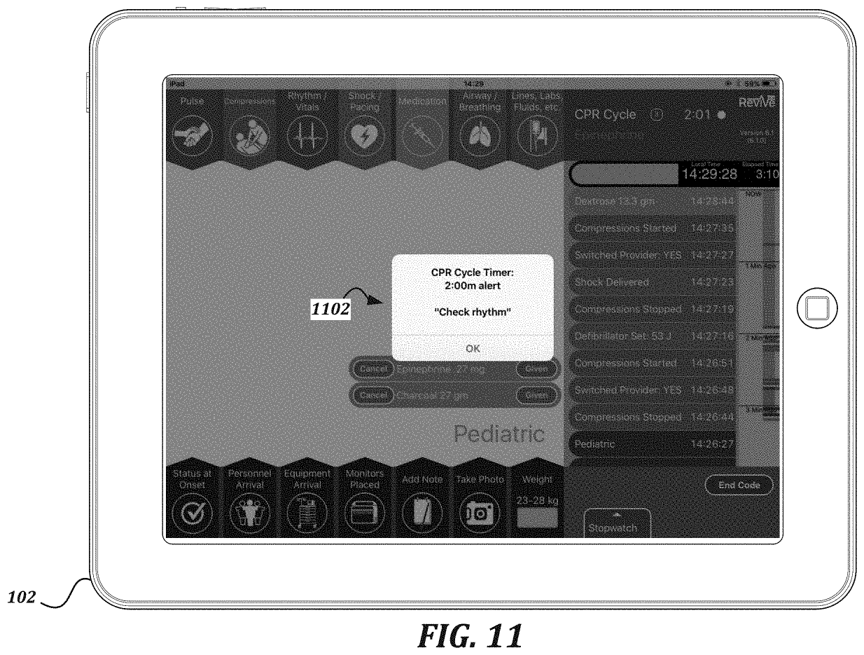

Otherwise, if it is determined that chest compressions are not being performed, then the result of decision block 208 is NO, and the method 200 advances directly to block 212 without executing the procedure in procedure block 210. At block 212, an alert generation module 108 of the ECMD 102 begins monitoring for timed events, and presents modal alerts upon detecting the occurrence of a timed event. One non-limiting example of a timed event is an amount of time for a round of chest compressions. Another non-limiting example of a timed event is an amount of time between doses of a medication. The presentation of the modal alert helps guide the caregivers to take action after the amount of time has passed according to a care guideline, thus removing the burden for tracking the amount of time that has passed from the user. FIG. 11 illustrates a non-limiting example embodiment of a modal alert 1102 presented by the alert generation module 108 upon the detection of the occurrence of a timed event.

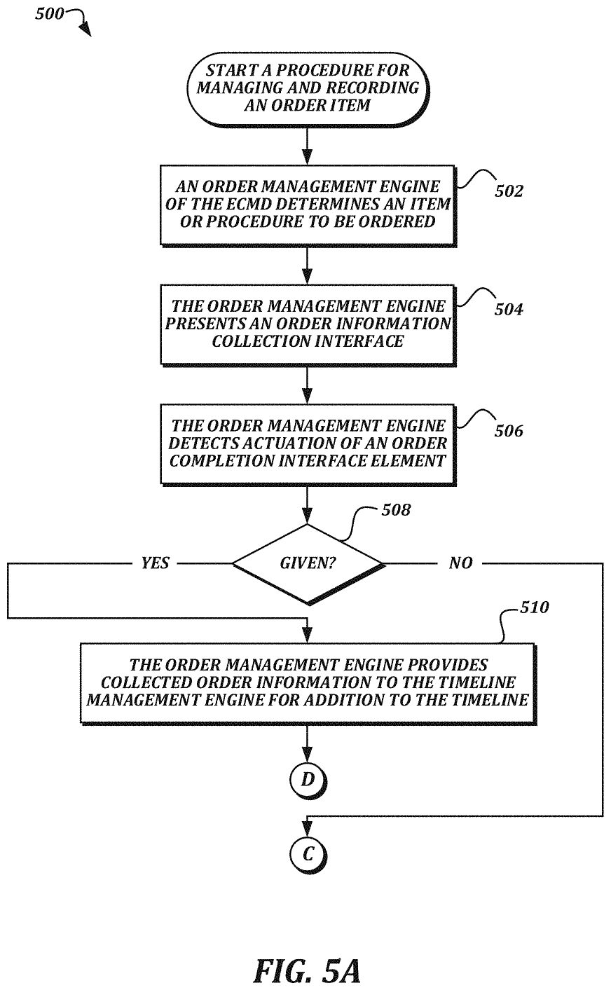

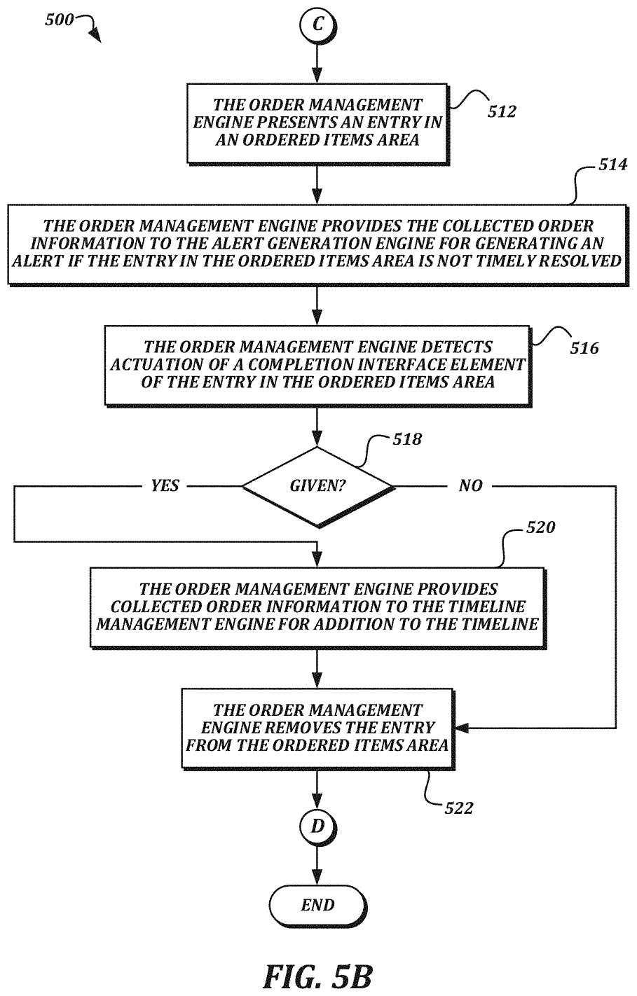

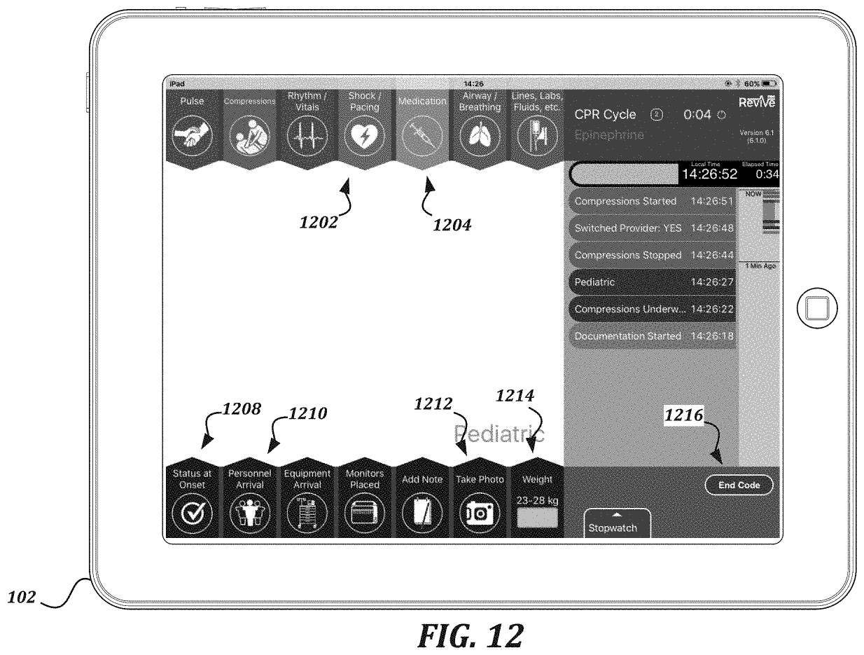

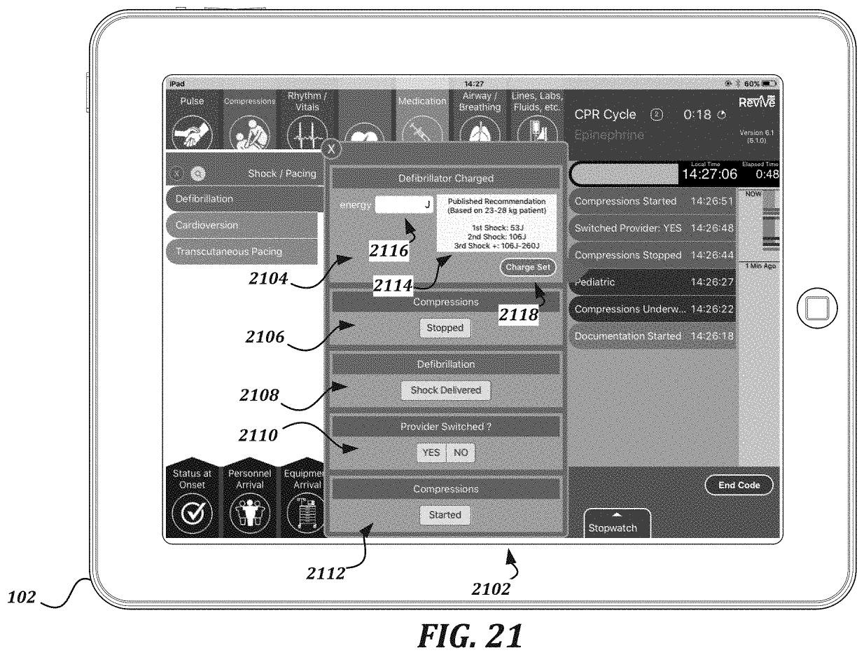

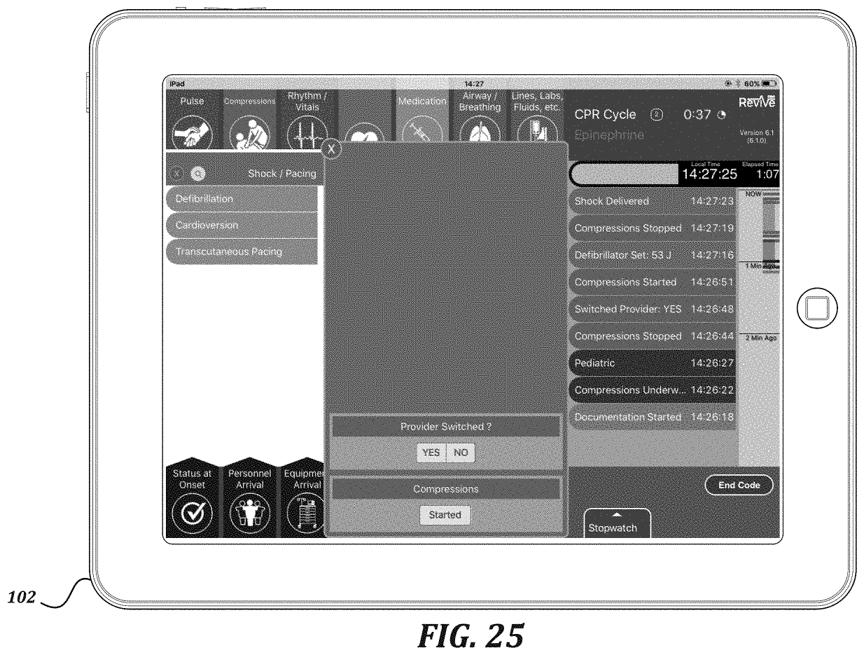

The method 200 then proceeds to a continuation terminal (“terminal A”). From terminal A (FIG. 2B), the method 200 proceeds to block 214, where the user interface management engine 122 monitors for actuation of a non-modal interface element. The interface that is presented includes a variety of non-modal interface elements that are usable to collect information about actions occurring during the event. FIG. 12 illustrates various non-limiting examples of non-modal interface elements that could be activated within the interface. Along the top edge of the interface, color-coded interface elements indicate care-related tasks that can be entered into the interface. For example, a shock/pacing interface element 1202 could be actuated in order to guide and record administration of a defibrillation shock, an external pacemaker, or other treatment (as illustrated in further detail below in FIGS. 21-27). As another example, a medication interface element 1204 could be actuated in order to guide and record administration of a medication (as illustrated in further detail below in FIGS. 5 and 32-38).

Along the bottom edge of the interface, non-colored interface elements indicate administrative information that can be entered when time is available. For example, a status-at-onset interface element 1208 may be actuated in order to display an interface for collecting non-urgent status information for the patient or a personnel arrival interface element 1210 may be actuated in order to record information about the care team. In an additional example, a photo capture interface element 1212 may be actuated in order to activate a camera of the ECMD 102. Once the camera is activated, a picture may be taken and added to the event record and/or timeline. One typical use for capturing a picture to be added to the event record may be to record an image of a chart (including but not limited to an ECG chart) in order to easily capture detailed information that is not readily entered in another format. A weight entry interface element 1214 may cause an interface to be presented similar to that illustrated in FIG. 10.

The weight entry interface element 1214 is an example of an interface element that presents, in a static format, information that was previously entered and needs to be readily available. As shown, the weight entry interface element 1214 includes the weight range that was entered, as well as a patch of color to quickly indicate the color code of the selected weight range. The text “pediatric” in the background also quickly indicates the previously entered weight information. Further description of the information collection interfaces provided by actuating some of these non-modal interface elements will be discussed below.

Upon detection of actuation of a non-modal interface element, the method 200 proceeds to decision block 216, where a determination is made regarding whether the non-modal interface element that was actuated was an end event interface element. FIG. 12 illustrates a non-limiting example of an end event interface element 1216. If it is determined that the actuated non-modal interface element was not an end event interface element, then the result of decision block 216 is NO, and the method 200 proceeds to block 218, where the user interface management engine 122 determines the actuated interface element and invokes an appropriate engine to process the desired action. For example, if the medication interface element was actuated, an order management engine may be invoked; if compressions interface element 1204 was actuated, the workflow presentation engine 112 may be invoked. Further details of actions that are performed in response to actuation of various types of interface elements are provide below. After invoking the appropriate engine, the method 200 returns to block 214.

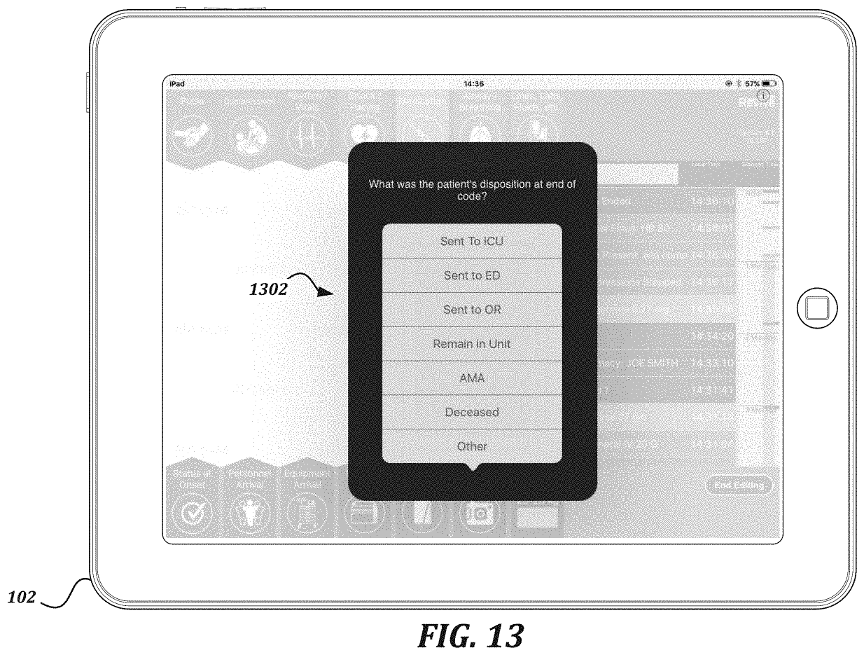

Returning to decision block 216, if it is determined that the actuated non-modal interface element was an end event interface element, then the result of decision block 216 is YES, and the method 200 proceeds to block 220, where an administrative data engine 114 of the ECMD 102 presents a modal dialog to collect a patient disposition. FIG. 13 illustrates a non-limiting example embodiment of an interface that includes a modal patient disposition dialog 1302. This information is collected in a modal dialog in order to improve the accuracy and completeness of the event record, since patient disposition is often missing from event records because it is not directly care-related. In some embodiments, the collected patient disposition value may be provided to the timeline management engine 106 to be entered as a final element in the timeline.

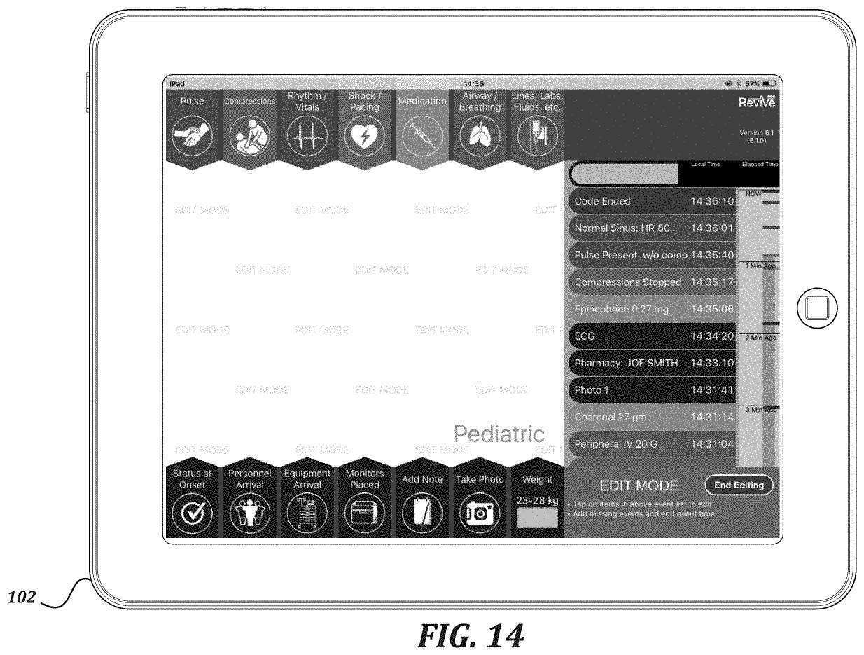

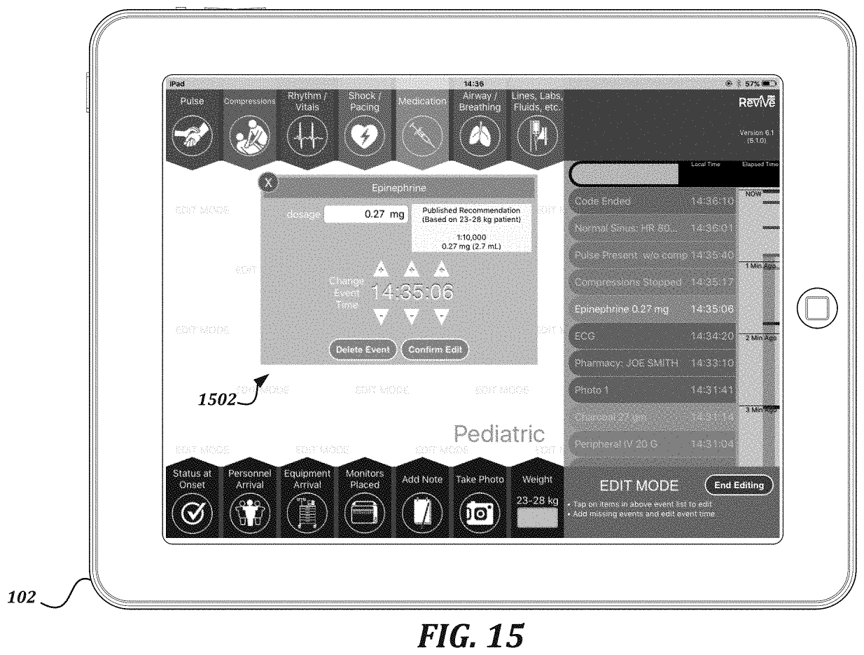

At block 222, the user interface management engine 122 places the interface in an edit mode, and the timeline management engine 106 monitors for edit requests on timeline events. FIG. 14 illustrates a non-limiting example embodiment of an interface in an edit mode. As shown, a background of the interface has been changed to display the text “edit mode” in repeated fashion in order to clearly indicate the change in mode. An edit request may be made by actuating any of the timeline entries. FIG. 15 illustrates a non-limiting example embodiment of editing a timeline entry within the edit mode. An event entry editing interface element 1502 is shown, which includes interface elements for changing the value associated with the timeline entry and for changing the time at which the timeline entry is placed in the timeline. Upon actuation of an interface element to confirm the edit (or to delete the timeline entry), the new values are recorded in the event record and the presentation of the timeline is changed. In some embodiments, the originally entered information is also retained in the event record to ensure the completeness of the records.

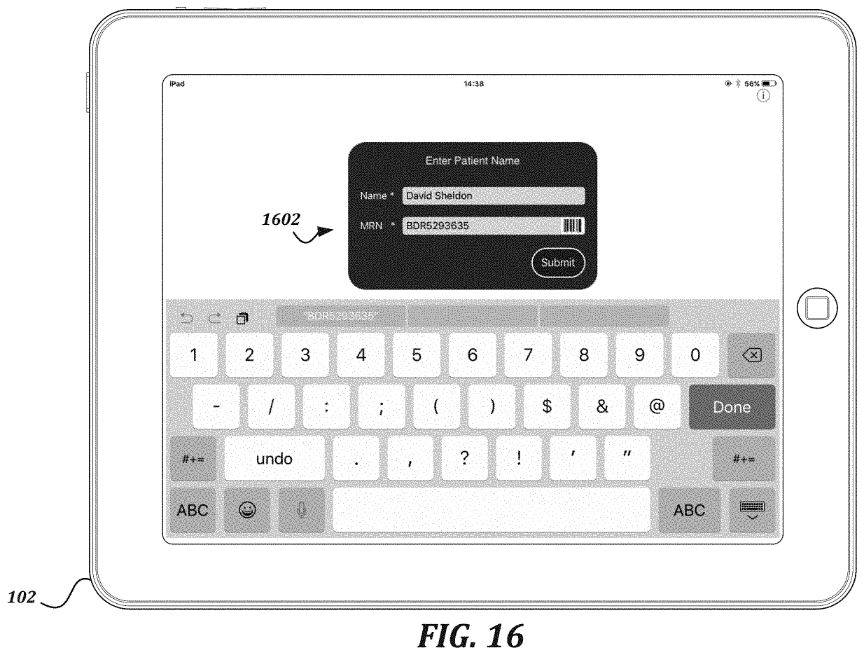

At block 224, the administrative data engine 114 presents a modal dialog to collect patient identifying information. FIG. 16 illustrates a non-limiting example embodiment of an interface configured to collect patient identifying information. As shown, a modal patent information interface element 1602 provides the ability to enter a patient name and a patient identifier. In some embodiments, the patient identifier may be scanned from a barcode bracelet, chart, or other barcode using the camera of the ECMD 102. If the patient identifying information was already input into another interface element earlier in the method 200, then the modal patient information interface element 1602 may be prepopulated with the proper information, or may be skipped. Collection of patient identifying information is as being performed after the event guidance and recording has been completed, because the identity of patient may not have an impact on treatment guidelines during treatment for cardiac or respiratory arrest, and so may not be critical for determining guidelines or other information to present. In some embodiments, the patient identifying information may be collected as care-dependent information in a modal interface element near the start of the method 200, which would allow specific information about the patient to be retrieved from the EMR server 92, including but not limited to allergies, medications that have been administered, and stored DNR information. However, the simplicity and streamlining of the start of the event guidance and recording method gained by removal of the step from the beginning of the method may be preferable. In some embodiments, the administrative data engine 114 may present additional modal interface elements to collect the identity of other individuals, such as members of the care team in attendance, the code leader, the recording user, and/or the like.

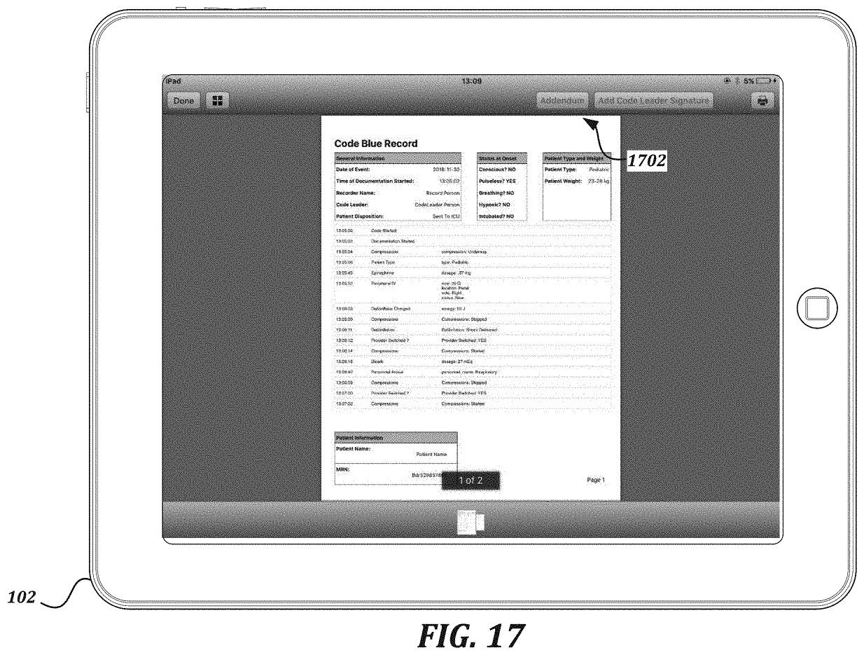

The method 200 then proceeds to a continuation terminal (“terminal B”). From terminal B (FIG. 2C), the method 200 proceeds to block 226, where the administrative data engine generates a summary document, and receives one or more annotations from a user. The summary document lists all of the information collected in the event record, and may also list changes to that information made in edit mode. The summary document may be considered an authoritative representation of the information collected by the ECMD 102 during the event. The annotating user may be a physician, a nurse, or another party responsible for running the event. In some embodiments, the annotating user cannot edit the summary document, but can add annotations intended to correct, change, or enhance information that was collected by the ECMD 102 during the event. FIG. 17 illustrates an example embodiment of a display of a summary document, along with an annotation interface element 1702 that allows the reviewing user to create an annotation for the event record.

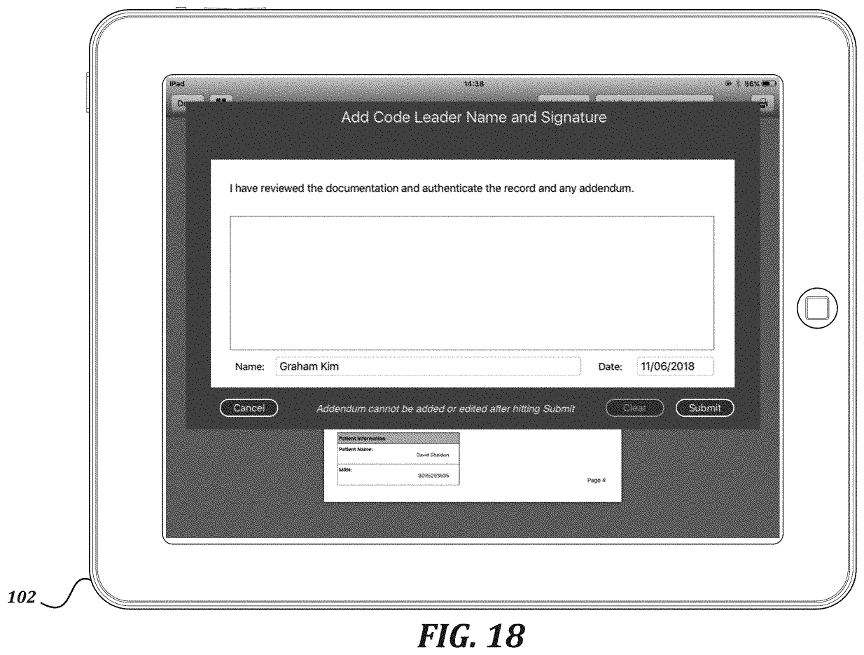

At block 228, the administrative data engine 114 obtains a signature from the user and finalizes an event record in an event data store 116 of the ECMD 102. In some embodiments, all of the information in the event record may be stored in the event data store 116 upon entry, and finalizing the event record may include generating an encrypted version of the event record that is protected from further edits or viewing by unauthorized parties, and/or generating a cryptographic hash of the event record to be used as proof that the record has not been altered. In some embodiments, a cryptographic signature may be applied to the event record during finalization. FIG. 18 illustrates a non-limiting example embodiment of an interface for receiving a signature from the user.

At block 230, a communication interface 110 of the ECMD 102 establishes a secure connection to an electronic medical record (EMR) server 92. In some embodiments, the secure connection may be established upon finalizing the event record. In some embodiments, the secure connection may not be established immediately upon finalizing the event record, but instead may wait until the ECMD 102 is within communication range of the network 90, or is otherwise able to establish the connection to the EMR server 92. In some embodiments, the secure connection may include an encrypted channel such as a TLS channel. In some embodiments, the secure connection may be protected by one or more passwords and/or multi-factor authentication. In some embodiments, the secure connection may include a physical connection between the ECMD 102 and the EMR server 92 (or a computing device capable of communicating with the EMR server 92). In some embodiments, instead of establishing a secure connection directly to the EMR server 92, the ECMD 102 may establish a secure connection to an intermediate computing device that collects finalized event records for final transmission to the EMR server 92.

At block 232, the communication interface 110 transmits the event record to the EMR server 92. This transmission may include the cryptographic hash, signature, or other value that can be used to verify that the finalized event record has not been altered. At block 234, in response to receiving an acknowledgement receipt from the EMR server 92 that indicates that the event record has been received, verified, and stored, the user interface management engine 122 deletes the event record from the event data store 116. Various benefits can be achieved by deleting the event record, such as increasing available space within the event data store 116, and reducing the potential for exposure of health-related information from old event records. The method 200 then proceeds to an end block and terminates.

FIG. 3 is a flowchart that illustrates an example embodiment of a procedure for managing and recording a treatment cycle item according to various aspects of the present disclosure. The procedure 300 is an example of a procedure that is suitable for use at procedure block 210 above, and is also suitable to describe the invocation of a cycle management engine 118 at block 218. In some embodiments, managing and recording a treatment cycle item provides an interface to guide and record the initiation of a treatment. After initiation of the treatment has been recorded, the amount of time since the initiation is tracked in order to present an alert at an appropriate time to initiate a new treatment cycle. A number of treatment cycles that have been performed may also be tracked. One example use is for guiding and recording administration of CPR, wherein some treatment guidelines state that a compression provider should be changed every two minutes for peak efficacy, and other treatment guidelines suggest treatment decisions that should be made based on how many cycles of CPR have been performed. Another example use is for guiding and tracking administration of medications such as epinephrine. Treatment guidelines state that doses of epinephrine should be provided ten minutes apart, and that the total number of doses administered should be tracked. The procedure 300 provides functionality for guiding and recording each of these actions.

From a start block, the procedure 300 advances to block 302, where the cycle management engine 118 detects the start of a treatment cycle. This may be in response to actuation of a treatment cycle item, as described in block 218, may be in response to another event that triggers the start of a treatment cycle such as at procedure block 212, or in response to any other suitable action. At block 304, the cycle management engine 118 starts a cycle timer for the treatment. The cycle timer tracks an elapsed time since the start of the current cycle being tracked. At block 306, the cycle management engine 118 increments a cycle counter for the treatment. The cycle counter tracks how many cycles have been performed, regardless of the duration of each cycle.

At block 308, the cycle management engine 118 provides the alert generation engine 108 with information to create at least one alert associated with the treatment cycle. In some embodiments, the information may include a time at which the cycle started and a cycle duration, from which the alert generation engine 108 can determine a time at which the alert should be presented. In some embodiments, the information may include a time at which the alert should be presented as determined by the cycle management engine 118, without including the time the cycle started or the cycle duration. In some embodiments, the information may include a message to be presented with the alert, a haptic pattern to present along with the alert, an alert sound to present along with the alert, or any other suitable information. At block 310, the cycle management engine 118 presents the cycle timer, the cycle counter, and a cycle time remaining indicator.

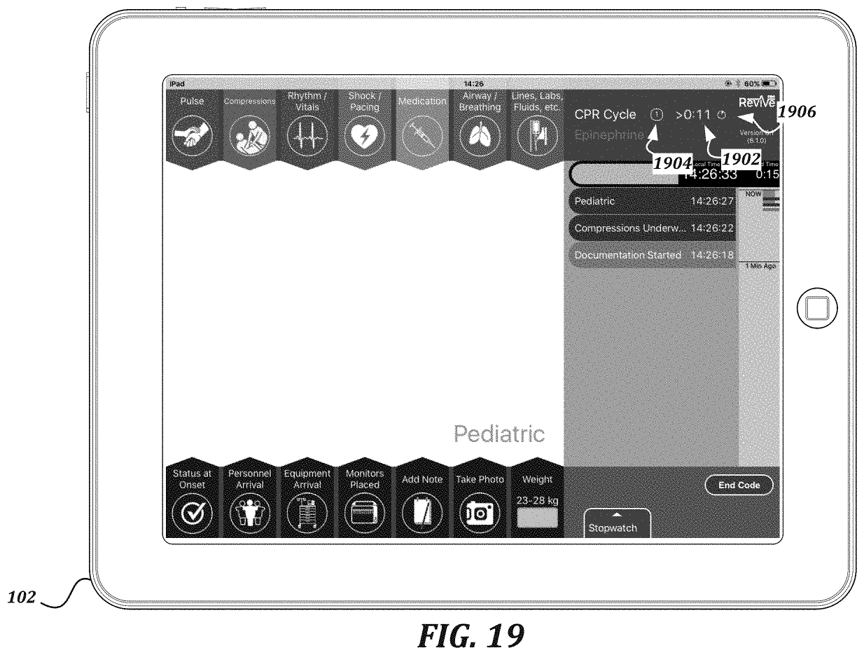

FIG. 19 illustrates a non-limiting example embodiment of an interface that includes a cycle timer 1902, cycle counter 1904, and cycle time remaining indicator 1906. As shown, the cycle time remaining indicator 1906 is shown as a pie to represent the time remaining in a compact, yet easily understandable format. In other embodiments, the cycle time remaining indicator 1904 could be a numeric time remaining value, a progress bar, or any other suitable format. In FIG. 19, the cycle timer 1902 indicates a “greater than” value. This may be shown to indicate, in a first cycle that was started before the care-dependent information was collected, that the cycle time is not exactly known because the actual treatment cycle being tracked may have started at some point before the method 200 began.

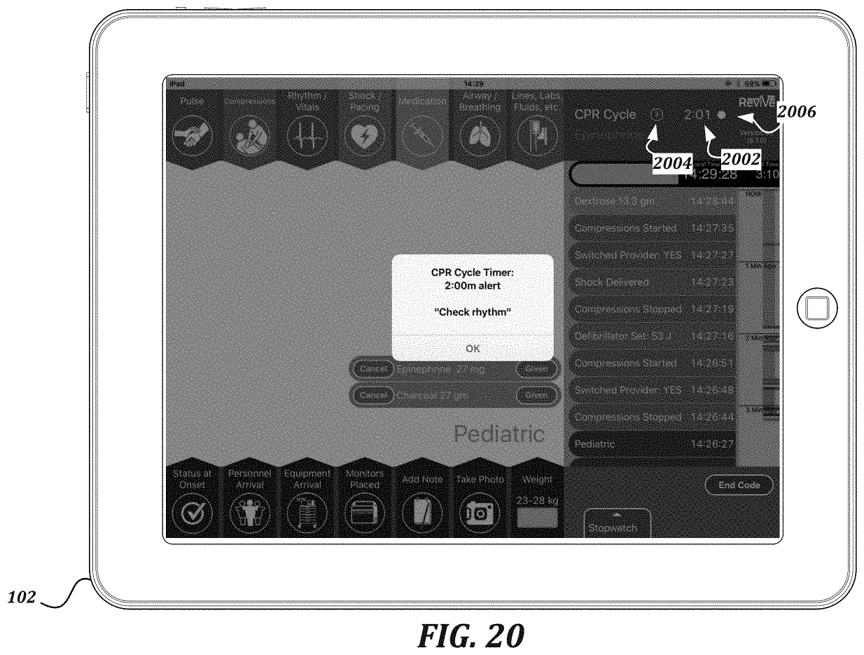

After block 310, the procedure 300 may remain idle, while other actions are being tracked. While the rest of the procedure 300 remains idle, the alert generation engine 108 may continue to monitor the cycle timer. Once the cycle timer expires (or the other conditions for the alert are met), the alert generation engine 108 presents an alert associated with the treatment cycle. The alert may include a message, a tone, a haptic pattern, or other information provided by the cycle management engine 118 to accompany the alert. In some embodiments, the alert may be presented in a modal interface element. FIG. 20 illustrates a non-limiting example embodiment of an interface that includes a modal alert presented by the alert generation engine 108 based on information provided by the cycle management engine 118. As shown, the cycle counter 2004 still indicates that the first cycle is ongoing, the cycle timer 2002 continues to track the elapsed time since the start of the current cycle, and the cycle time remaining indicator 2006 shows that the cycle time has been completed.

The procedure 300 then advances to a decision block 314, where a determination is made regarding whether a subsequent cycle is starting. In some embodiments, a subsequent cycle may start in response to selection or detection of another interface element that starts the next cycle. If a subsequent cycle is starting, then the result of decision block 314 is YES, and the procedure 300 returns to block 304. Otherwise, if a subsequent cycle is not starting, then the result of decision block 314 is NO, and the procedure 300 advances to an end block and terminates. In some embodiments, instead of terminating, the procedure 300 may return to block 310, and may simply continue to present the cycle timer, the cycle counter, and an expired cycle time remaining indicator until a subsequent cycle begins, whether or not another alert is generated at block 312. In some embodiments, an additional alert may be generated after a second amount of time elapses if the next cycle has not been started.

FIG. 4 is a flowchart that illustrates an example embodiment of a procedure for managing and recording a workflow item according to various aspects of the present disclosure. The procedure 400 is an example of a procedure suitable for invoking the workflow presentation engine 112 at block 218 above. A workflow item combines guidance and data collection for a task that has a number of steps that should be performed according to treatment guidelines. One non-limiting example of such a task would be guiding actions and collecting information regarding delivery of an electric shock such as a defibrillation shock to a patient, which is best performed by following a specific set of steps that may otherwise be difficult to follow and record accurately during the frenetic activity of an emergency care event. In addition to providing guidance, another beneficial aspect provided by the workflow presentation engine 112 is that the completion of each workflow step can be automatically added to the timeline, and so an exact amount of time between each workflow step can be recorded for later analysis.

From a start block, the procedure 400 advances to block 402, where a workflow presentation engine 112 of the ECMD 102 determines a list of workflow steps associated with the workflow item, and at block 404, the workflow presentation engine 112 presents the list of workflow steps. In some embodiments, the workflow presentation engine 112 may be pre-programmed with the list of workflow steps associated with each supported workflow item. In some embodiments, the workflow presentation engine 112 may retrieve the list of workflow steps from a computer-readable medium of the ECMD 102. In some embodiments, each workflow step in the list of workflow steps includes one or more interface elements usable to complete the workflow step, and may also include one or more of a description of the step and one or more interface elements for collecting information associated with the workflow step.

FIG. 21 illustrates a non-limiting example embodiment of an interface that is presenting a list of workflow steps 2102. The list of workflow steps 2102 includes a first workflow step 2104, a second workflow step 2106, a third workflow step 2108, a fourth workflow step 2110, and a fifth workflow step 2112. As shown, the first workflow step 2104 includes information explaining specific treatment guidelines 2114 that may be based on the care-dependent information gathered earlier, an input interface element 2116 for entry of a value that was actually used/ordered, and a completion interface element 2118. In some embodiments, if an entry is made in the input interface element 2116 that is outside of the guidelines 2114, an alert may be presented by the alert generation engine 108. In some embodiments, only the first workflow step 2104 may include interface elements that are enabled, and the remainder of the workflow steps 2106-2112 may only include interface elements that are disabled, in order to force resolution of the first workflow step 2104 before moving on to other workflow steps. Naturally, the list of workflow steps 2102 illustrated in FIG. 21 is an example only, and embodiments of the present disclosure may include more, fewer, or different workflow steps in a list of workflow steps.

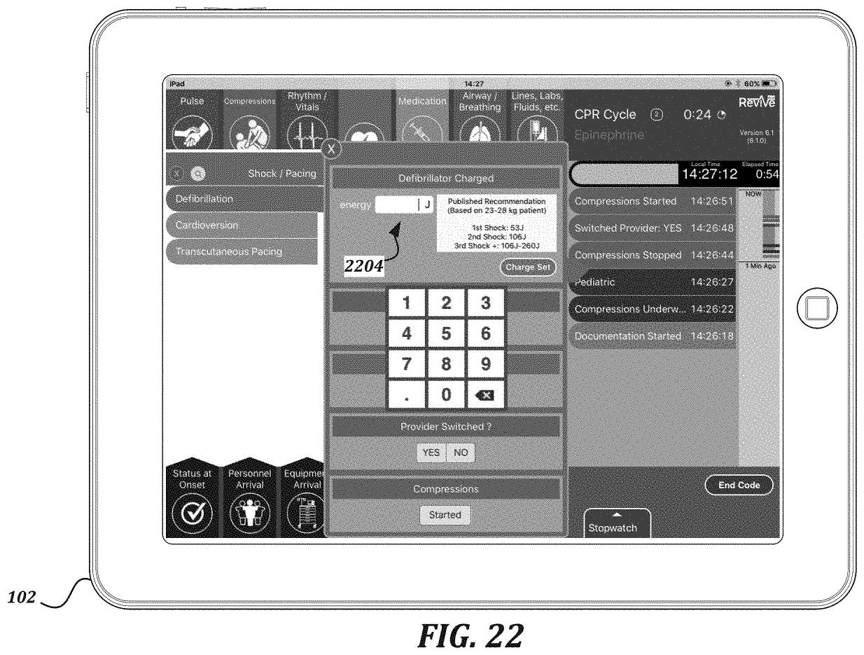

At optional block 406, the workflow presentation engine 112 receives information in the top workflow step and associates the information with a completion interface element. In some embodiments, the information may be received in an input interface element, such as input interface element 2116 illustrated in FIG. 21. Block 406 is illustrated as optional because for some workflow steps, additional information may not be collected or received. FIG. 22 is a non-limiting example embodiment of an interface that is collecting information in first workflow step 2204, which is the top workflow step.

At block 408, the workflow presentation engine 112 detects actuation of a completion interface element for the top workflow step. In FIG. 21, the top workflow step is the first workflow step 2104, and the actuated completion interface element is the completion interface element 2118. At block 410, the workflow presentation engine 112 provides information associated with the completion interface element to the timeline management engine 106 for addition to the timeline. The information associated with the completion interface element may include an identity of the completion interface element or some other value associated with the completion interface element. This is particularly relevant when the workflow step includes more than one completion interface element for indicating different resolutions of the workflow step (such as the fourth workflow step 2110 illustrated in FIG. 21.

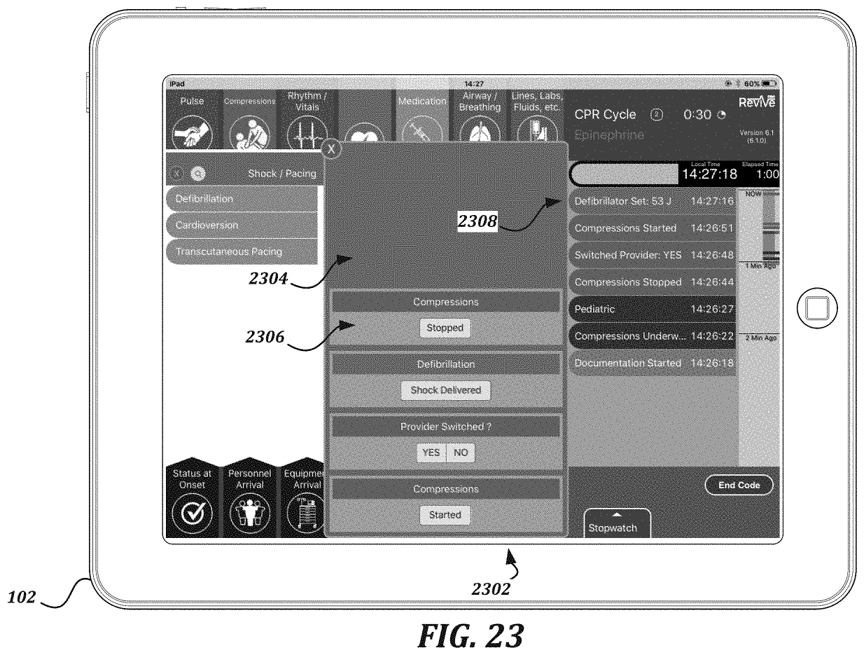

At block 412, the workflow presentation engine 112 removes the top workflow step from the list of workflow steps. FIG. 23 illustrates a non-limiting example embodiment of an interface wherein the first workflow step has been removed. As shown, the area 2304 where the first workflow step was formerly presented is now empty, and a new timeline event 2308 has been added. The timeline event 2308 includes the information that was entered into the first workflow step. As shown, the second workflow step 2306 is now the top workflow step in the list of workflow steps 2302. In some embodiments, instead of showing empty space where the completed workflow step was presently displayed, the list of workflow steps 2302 may be resized to display only the remaining workflow steps, though leaving the empty space may be beneficial in that it provides a visual indicator of how many workflow steps have already taken place.

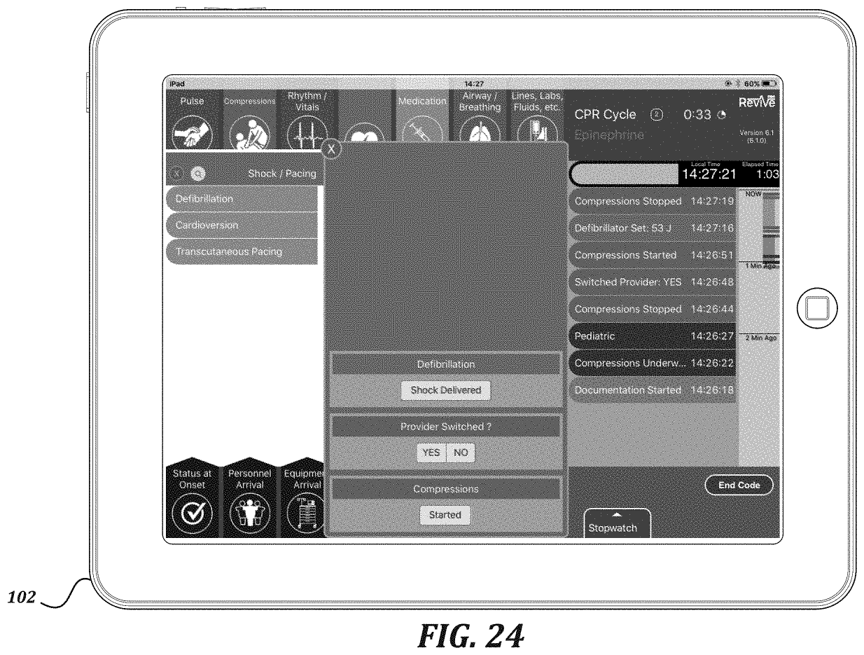

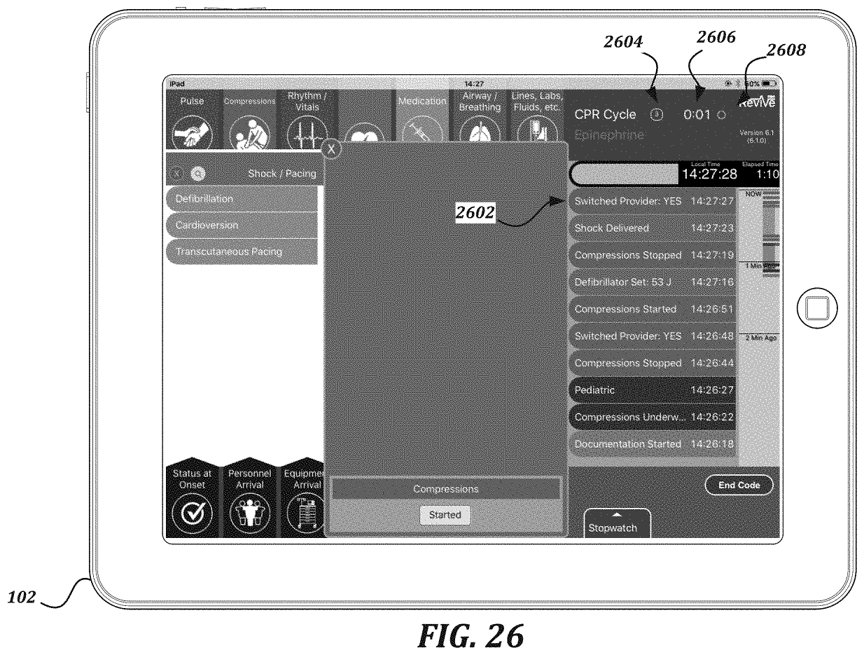

The procedure 400 then advances to a decision block 414, where a determination is made regarding whether more steps are present in the list of workflow steps after removing the top workflow step. If more workflow steps remain, then the result of decision block 414 is YES, and the procedure 400 returns to optional block 406 to process the new top workflow step. FIGS. 24-27 illustrate non-limiting example embodiments of the previously illustrated workflow steps being processed through blocks 406-414. In FIG. 26, the timeline event 2602 added for the completion of the fourth workflow step includes an identification of the completion interface element that was actuated to complete the fourth workflow step. Further, because the completion interface element indicated that a CPR provider changed, the cycle management engine 118 detected that as the start of a new cycle, and so the cycle count 2604 has been incremented, and the cycle timer 2606 and the cycle time remaining indicator 2608 have been reset.

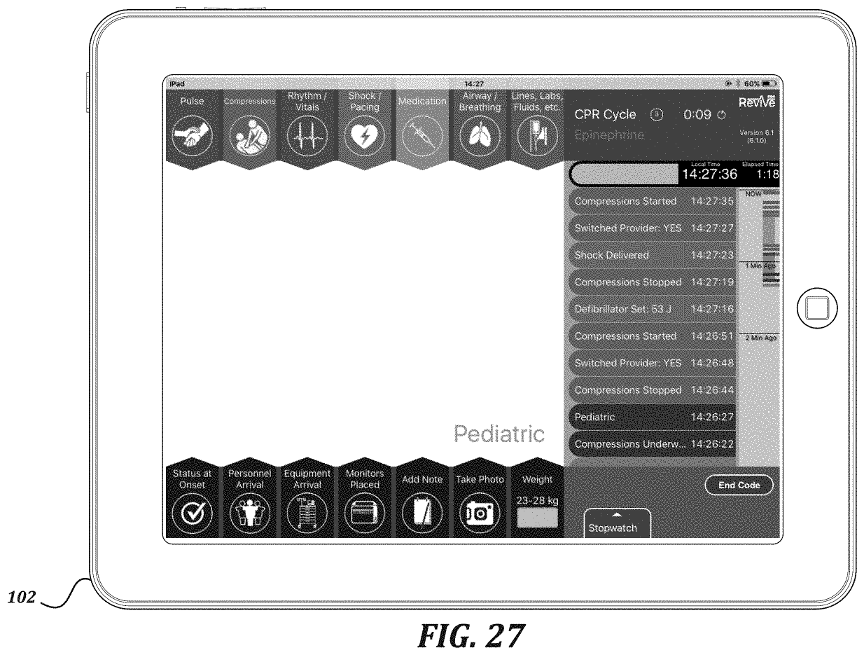

Returning to decision block 414, if no more workflow steps remain, then the result of decision block 414 is NO, and the procedure 400 advances to block 416, where the workflow presentation engine 112 closes the list of workflow steps. This is shown in FIG. 27. The procedure 400 then ends.

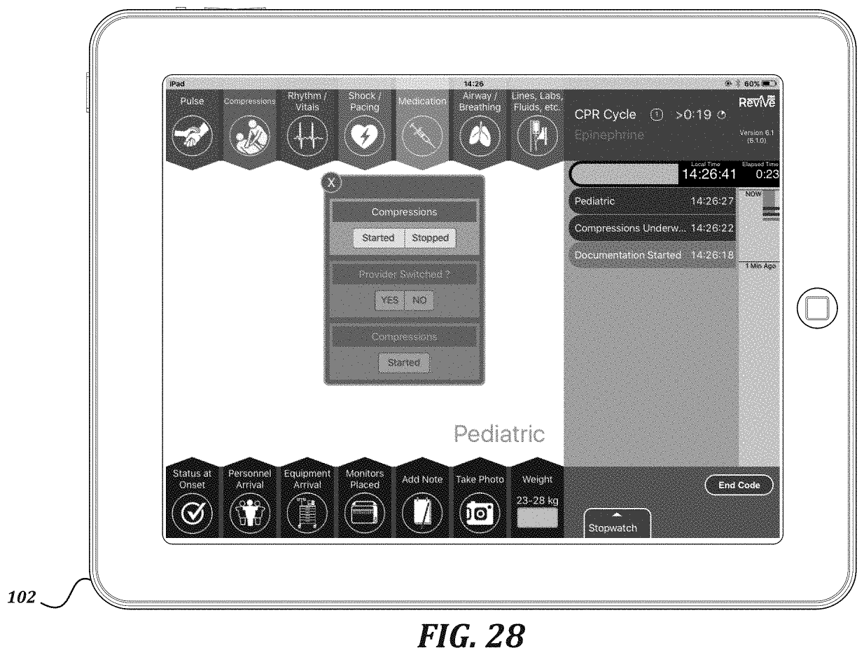

Though FIGS. 21-27 illustrate a workflow item that guides and records steps related to delivery of a defibrillation shock, this is only one non-limiting example of a workflow item. In some embodiments, other procedures that involve following and recording a set of steps may be guided and tracked by a workflow item. Another example of a suitable task to guide and record using a workflow item is a technique for switching CPR compression providers. When compression providers are switched, it is important to track the timing of each step, so a workflow item is appropriate for tracking such an event. FIGS. 28-31 illustrate a non-limiting example embodiment of interfaces for processing a workflow item for changing compression providers. The interfaces include similar features to those illustrated in FIGS. 21-27, including the increment of the cycle counter and the reset of the cycle timer and cycle time remaining indicator, and so are not described in further detail here.

FIGS. 5A-5B are a flowchart that illustrates an example embodiment of a procedure for managing and recording an order item according to various aspects of the present disclosure. The procedure 500 is an example of a procedure suitable for invoking the order management engine 120 at block 218 above. In some embodiments, an order item records an order for a medication, procedure, or other treatment, including details about what was ordered. The order item also records when the ordered item was actually administered. One problem that often occurs is that there is a lag between the ordering of the item by a physician or other caregiver and the actual administration of the item, and some ordered items may either never be administered or may be administered too late to have significant effect. To help address these problems, the procedure 500 actively provides alerts if it is determined that too much time has passed between ordering an item and the administration of the item.

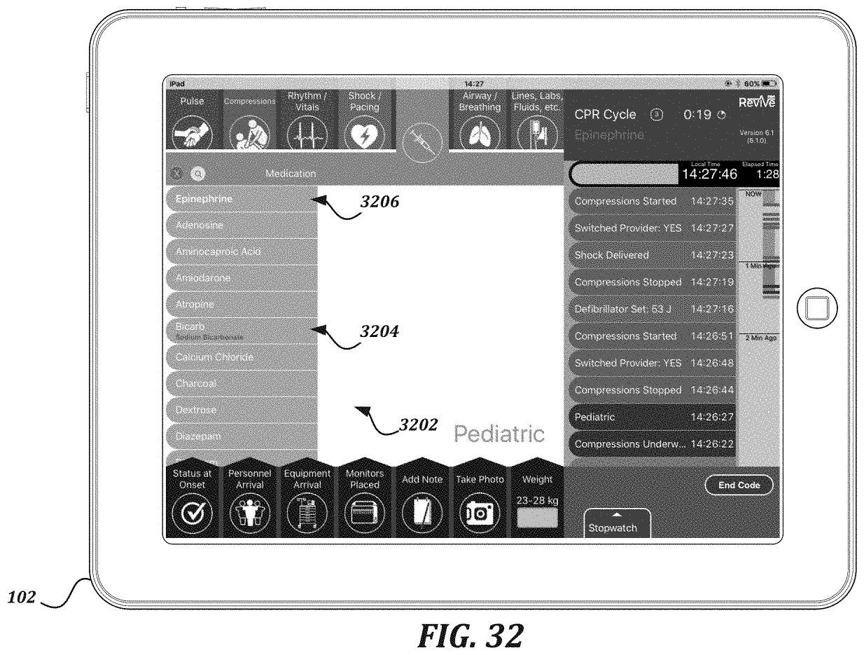

From a start block (FIG. 5A), the procedure 500 advances to block 502, where an order management engine 120 of the ECMD 102 determines an item or procedure to be ordered. In some embodiments, the determination may be based on a user selection from a list of possible items or procedures. FIG. 32 illustrates a non-limiting example embodiment of an interface that includes a presentation of a list of medication items 3202 that could be ordered. In some embodiments, the list of medication items 3202 may be scrollable by swiping up or down on the list. In some embodiments, the list of medication items 3202 may be organized alphabetically. In some embodiments, multiple entries in the list of medication items 3202 may be provided for items that have a formal name and a colloquial name. For example, the medication item 3204 for “bicarb,” which is a colloquial name, also shows its formal name of “sodium bicarbonate.” In some embodiments, some frequently selected items may be pinned to the top of the list of medication items 3202 regardless of alphabetization. For example, the medication item 3206 for epinephrine appears at the top of the list of medication items 3202.

At block 504, the order management engine 120 presents an order information collection interface. In some embodiments, the order information collection interface may collect information regarding details for the ordered item, including but not limited to one or more of an amount or dosage, a size, and an administration location. In some embodiments, the order information collection interface may include one or more completion interface elements that can be actuated to complete the order. In some embodiments, an ordered completion interface element may be used to indicate that the item was ordered but not yet administered, and a given completion interface element may be used to indicate that the item was both ordered and given.

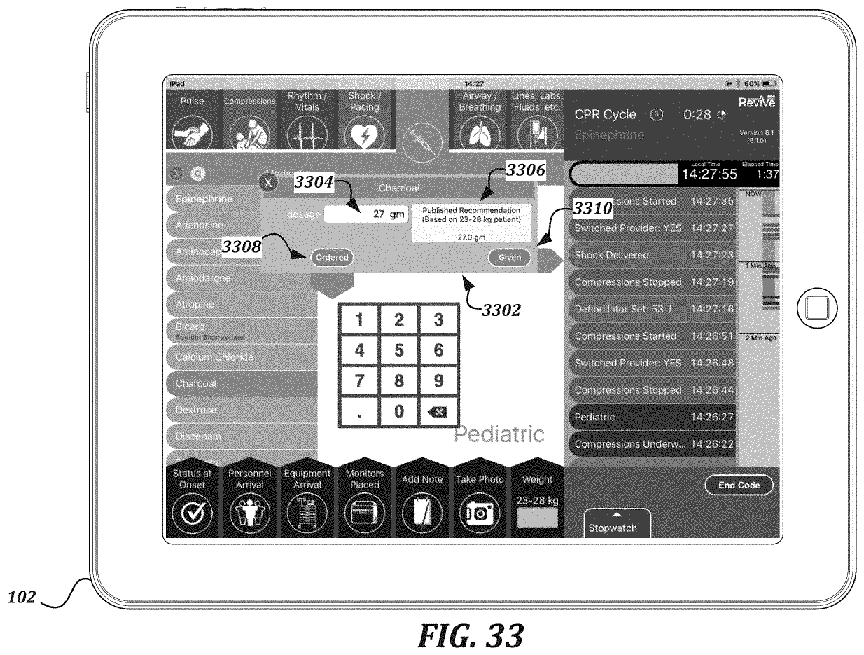

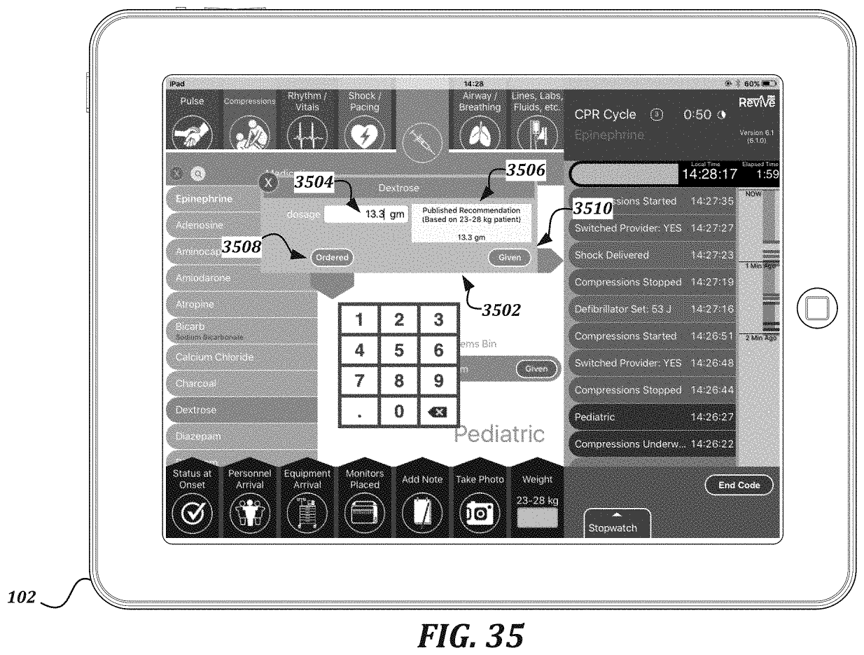

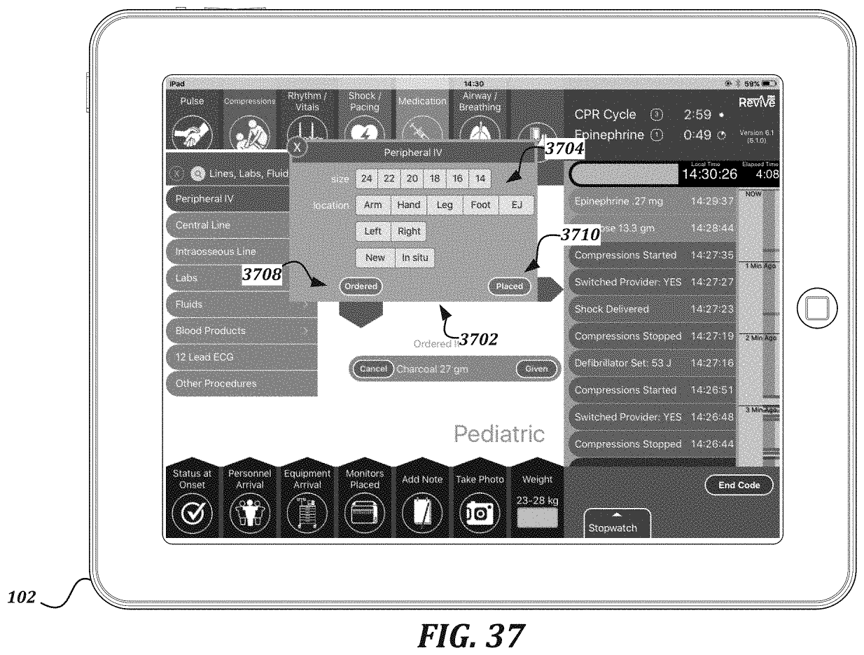

FIG. 33 illustrates a non-limiting example embodiment of an interface that includes an order information collection interface 3302. The order information collection interface 3302 includes an entry interface element 3304 to allow an ordered dosage to be input by the user, and a recommendation interface element 3306 that presents a guideline based on the care-dependent information. The order information collection interface 3302 also includes an ordered completion interface element 3308 and a given completion interface element 3310. FIG. 35 illustrates another non-limiting example interface with an order information collection interface 3502 for another type of medication that uses an entry interface element 3504, a recommendation interface element 3306, an ordered completion interface element 3508, and a given completion interface element 3510. FIG. 37 illustrates another non-limiting example interface with an order information collection interface 3702 for a peripheral IV. The order information collection interface 3702 includes a set of interface elements 3704 for collecting information about the size and placement of the IV, an ordered completion interface element 3708 and a given completion interface element 3710.

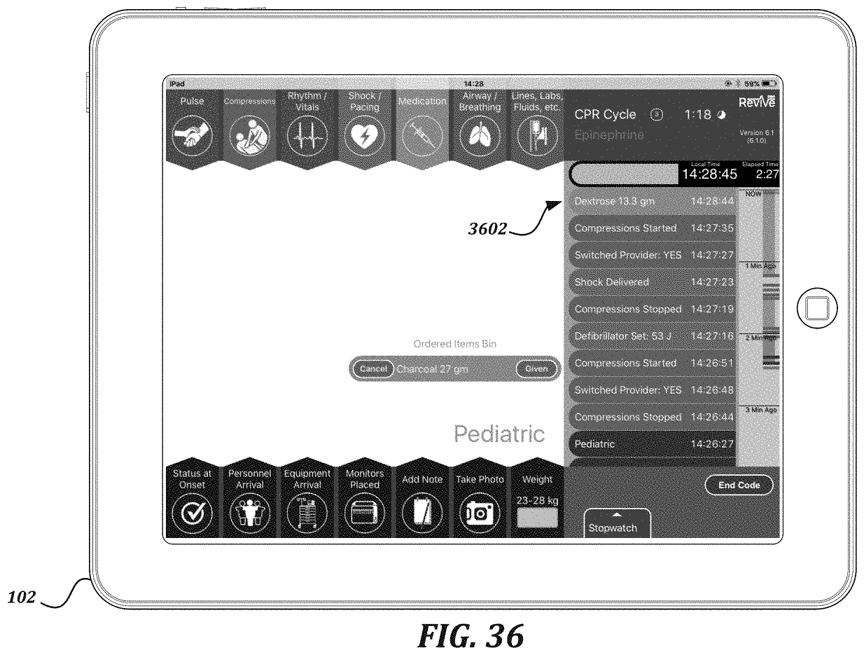

At block 506, the order management engine 120 detects actuation of an order completion interface element, and at decision block 508, a determination is made regarding whether the actuation indicates that the treatment was ordered or given. If the actuation indicates that the treatment was given, then the result of decision block 508 is YES, and the procedure 500 advances to block 510, where the order management engine 120 provides collected order information to the timeline management engine 106 for addition to the timeline. As shown in FIG. 36, the given completion interface element 3510 of FIG. 35 was actuated, and so a timeline entry 3602 (FIG. 36) was created to record the administration of the treatment. The procedure 500 then advances to a continuation terminal (“terminal D”).

Returning to decision block 508, if the actuation indicates that the treatment was ordered only (and not given), then the result of decision block 508 is NO, and the procedure 500 advances to another continuation terminal (“terminal C”). From terminal C (FIG. 5B), the procedure 500 advances to block 512, where the order management engine 120 presents an entry in an ordered items area. In some embodiments, the order management engine 120 may also store an entry in the event record that indicates a time when the order was entered, even if a corresponding visual entry in the timeline is not created.

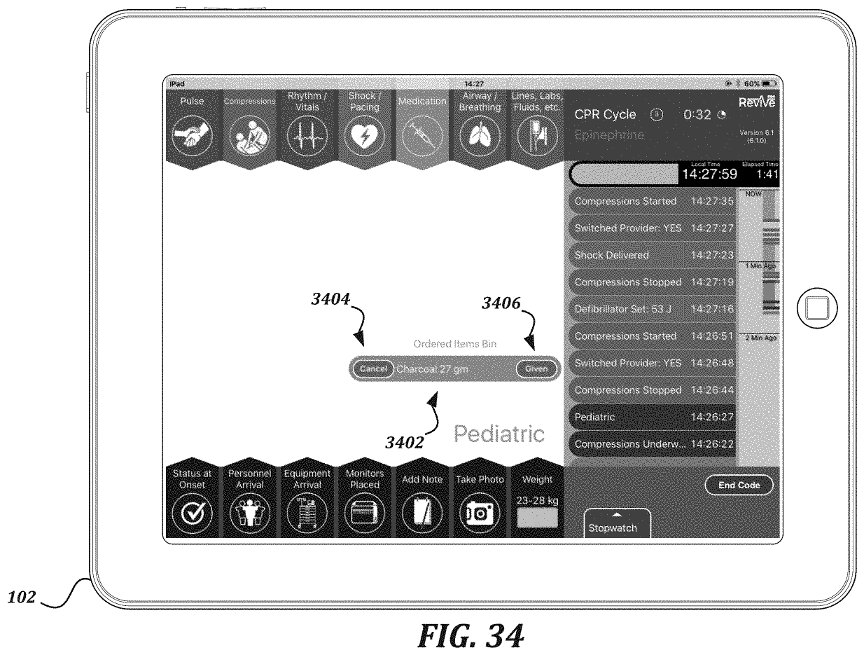

FIG. 34 illustrates a non-limiting example embodiment of an interface presented after the item from FIG. 33 was completed with the ordered completion interface element 3308. The interface includes an entry 3402 in an ordered items area that represents. The entry 3402 includes the identity of the ordered item and the ordered amount. The entry 3402 also includes a cancel completion interface element 3404 and a given completion interface element 3406.

At block 514, the order management engine 120 provides the collected order information to the alert generation engine 108 for generating an alert if the entry in the ordered items area is not timely resolved. The information provided to the alert generation engine 108 may include an elapsed time or a time of day at which an alert should be presented if the entry is still in the ordered items area without having been resolved. In some embodiments, the elapsed time or time of day may be determined by the order management engine 120 based on a type of the item (e.g., some items may be allowed to wait in the ordered items bin longer than others. In some embodiments, a standard timer length may be used for items that are not associated with a specialized timer.

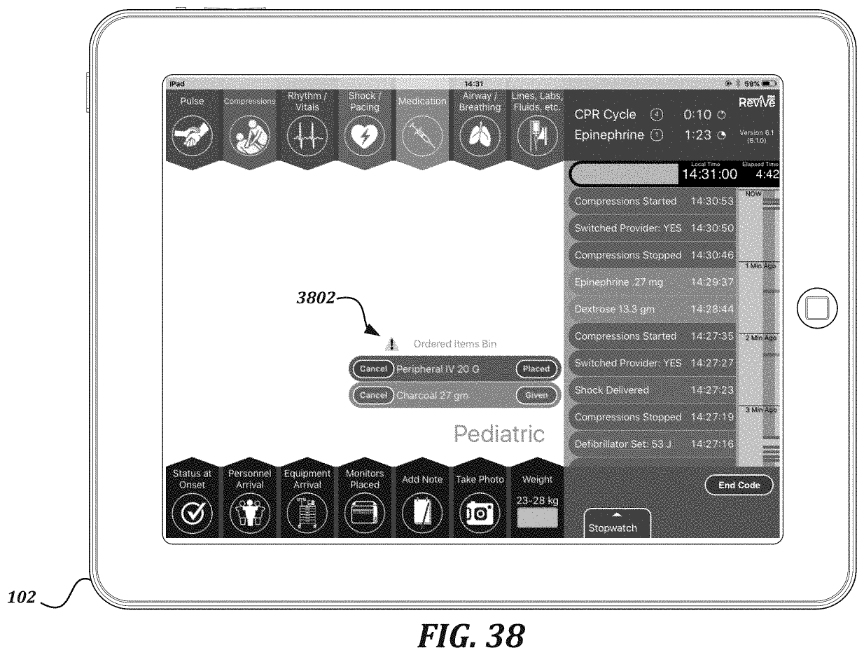

FIG. 38 illustrates a non-limiting example embodiment of an interface that includes an alert for the ordered items area. The alert 3802 is presented when the alert generation engine 108 has determined that at least one of the entries in the ordered items area has been presented for too long. In some embodiments, the alert may be presented on or next to the stale entry itself. In some embodiments, the alert 3802 may be animated, may flash, or may be presented as a modal dialog instead of an icon. The alert 3802 may continue to be presented until a completion interface element of the stale entry is actuated.

At block 516, the order management engine 120 detects actuation of a completion interface element of the entry in the ordered items area, and at decision block 518, a determination is made regarding whether the given completion interface element was actuated or the cancel completion interface element was actuated. If the given completion interface element was actuated, then the result of decision block 518 is YES, and the procedure 500 advances to block 520, where the order management engine 120 provides collected order information to the timeline management engine 106 for addition to the timeline. This is similar to the actions performed at block 510, and so is not described in further detail here for the sake of brevity. The procedure 500 then advances to block 522. At decision block 518, if the cancel completion interface element was actuated, then the result of decision block 518 is NO, and the procedure 500 advances directly to block 522.

At block 522, the order management engine 120 removes the entry from the ordered items area. If the entry was canceled instead of being given, the order management engine 120 may also add an event to the timeline (or a non-displayed event to the event record) to record the cancellation of the order. The procedure 500 then advances through terminal D to an end block and exits.

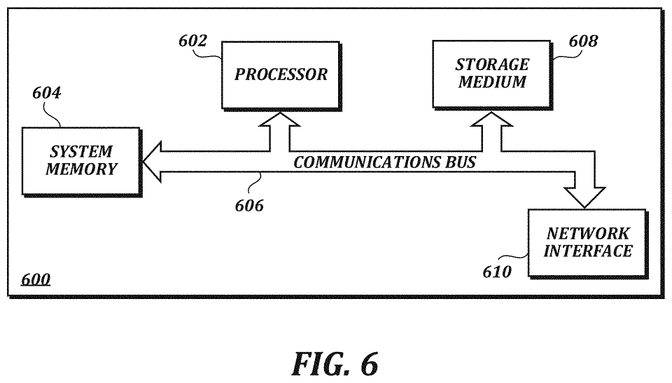

FIG. 6 is a block diagram that illustrates aspects of a representative computing device 600 appropriate for use with embodiments of the present disclosure. While FIG. 6 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Moreover, those of ordinary skill in the art and others will recognize that the computing device 600 may be any one of any number of currently available or yet to be developed devices.

In its most basic configuration, the computing device 600 includes at least one processor 602 and a system memory 604 connected by a communication bus 606. Depending on the exact configuration and type of device, the system memory 604 may be volatile or nonvolatile memory, such as read only memory (“ROM”), random access memory (“RAM”), EEPROM, flash memory, or similar memory technology. Those of ordinary skill in the art and others will recognize that system memory 604 typically stores data and/or program modules that are immediately accessible to and/or currently being operated on by the processor 602. In this regard, the processor 602 may serve as a computational center of the computing device 600 by supporting the execution of instructions.

As further illustrated in FIG. 6, the computing device 600 may include a network interface 610 comprising one or more components for communicating with other devices over a network. Embodiments of the present disclosure may access basic services that utilize the network interface 610 to perform communications using common network protocols. The network interface 610 may also include a wireless network interface configured to communicate via one or more wireless communication protocols, such as Wi-Fi, 2G, 3G, LTE, WiMAX, Bluetooth, and/or the like.

In the exemplary embodiment depicted in FIG. 6, the computing device 600 also includes a storage medium 608. However, services may be accessed using a computing device that does not include means for persisting data to a local storage medium. Therefore, the storage medium 608 depicted in FIG. 6 is represented with a dashed line to indicate that the storage medium 608 is optional. In any event, the storage medium 608 may be volatile or nonvolatile, removable or non-removable, implemented using any technology capable of storing information such as, but not limited to, a hard drive, solid state drive, flash memory, CD-ROM, DVD, or other disk storage, magnetic cassettes, magnetic tape, magnetic disk storage, and/or the like.

As used herein, the term “computer-readable medium” includes volatile and non-volatile and removable and non-removable media implemented in any method or technology capable of storing information, such as computer readable instructions, data structures, program modules, or other data. In this regard, the system memory 604 and storage medium 608 depicted in FIG. 6 are merely examples of computer-readable media. Computer-readable media can be used to store data for use by programs.

Suitable implementations of computing devices that include a processor 602, system memory 604, communication bus 606, storage medium 608, and network interface 610 are known and commercially available. For ease of illustration and because it is not important for an understanding of the claimed subject matter, FIG. 6 does not show some of the typical components of many computing devices. In this regard, the computing device 600 may include input devices, such as a keyboard, keypad, mouse, microphone, touch input device, touch screen, tablet, and/or the like. Such input devices may be coupled to the computing device 600 by wired or wireless connections including RF, infrared, serial, parallel, Bluetooth, USB, or other suitable connections protocols using wireless or physical connections. Similarly, the computing device 600 may also include output devices such as a display, speakers, printer, etc. Since these devices are well known in the art, they are not illustrated or described further herein.

As will be appreciated by one skilled in the art, the specific routines described above in the flowcharts may represent one or more of any number of processing strategies such as event-driven, interrupt-driven, multi-tasking, multi-threading, and the like. As such, various acts or functions illustrated may be performed in the sequence illustrated, in parallel, or in some cases omitted. Likewise, the order of processing is not necessarily required to achieve the features and advantages, but is provided for ease of illustration and description. Although not explicitly illustrated, one or more of the illustrated acts or functions may be repeatedly performed depending on the particular strategy being used. Further, these FIGURES may graphically represent code to be programmed into a computer-readable storage medium associated with a computing device.

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

The embodiments of the invention in which an exclusive property or privilege is claimed are defined as follows:

1. A mobile computing device for managing and logging actions during an emergency care event, the mobile computing device comprising:
a touch-enabled display device;
one or more processors;
an event data store;
a communication interface; and
a non-transitory computer-readable medium having computer-executable instructions stored therein that, in response to execution by the one or more processors, cause the computing device to:
present a first modal interface element to collect information that defines at least one care guideline, and close the modal interface element after collecting the information;
in response to detecting actuation of an event-initiating interface element, present a non-modal interface to manage and record event information, wherein content of the at non-modal interface element is determined by the at least one care guideline; and
store collected information in an event record in the event data store.

2. The mobile computing device of claim 1, wherein the information that defines at least one care guideline includes at least one of a patient weight, an indication of whether compressions are underway, and an indication of whether or not the patient is pediatric.

3. The mobile computing device of claim 1, wherein the instructions further cause the computing device to:
in response to detecting actuation of an end interface element:
present a second modal interface element to collect administrative information;
establish a connection via the communication interface to an electronic medical record (EMR) server; and
transmit the event record to the EMR server.

4. The mobile computing device of claim 1, wherein the instructions further cause the computing device to track a treatment cycle.

5. The mobile computing device of claim 4, wherein tracking a treatment cycle comprises:
starting a treatment cycle timer;
incrementing a treatment cycle counter; and
presenting the treatment cycle counter, the treatment cycle timer, and a treatment cycle time remaining indicator that is based on the treatment cycle timer.

6. The mobile computing device of claim 5, wherein tracking the treatment cycle further comprises presenting a modal alert upon detecting that the treatment cycle timer has reached a predetermined time.

7. The mobile computing device of claim 6, wherein tracking the treatment cycle further comprises:
detecting the start of a subsequent treatment cycle;
incrementing the treatment cycle counter; and
resetting the treatment cycle timer and the treatment cycle time remaining indicator.

8. The mobile computing device of claim 6, wherein the treatment cycle is for a dose of epinephrine, and wherein the modal alert indicates an amount of time that has elapsed since the previous epinephrine dose.

9. The mobile computing device of claim 6, wherein the treatment cycle is a cardiopulmonary resuscitation (CPR) cycle, and wherein the modal alert includes a reminder to switch compression providers.

10. The mobile computing device of claim 1, wherein presenting the non-modal interface includes presenting a workflow interface.

11. The mobile computing device of claim 10, wherein presenting the workflow interface includes:
presenting an ordered list of workflow steps;
detecting actuation of a completion interface element of a first workflow step in the ordered list of workflow steps;
inserting information associated with the first workflow step into the event record; and
removing the first workflow step from the ordered list of workflow steps.

12. The mobile computing device of claim 1, wherein presenting the non-modal interface includes presenting an order information collection interface.

13. The mobile computing device of claim 12, wherein presenting the order information collection interface includes:
receiving a selection of an ordered item or procedure;
presenting an order information collection interface that includes a given completion interface element and an ordered completion interface element;
in response to detecting actuation of the given completion interface element, entering information in the order information collection interface into the event record; and
in response to detecting actuation of the ordered completion interface element, presenting an ordered item interface element.

14. The mobile computing device of claim 13, wherein the instructions further cause the computing device to:
detect an ordered item interface element that has been presented for at least a threshold amount of time without being resolved; and
presenting an alert indicating that the ordered item interface element has not been resolved.

15. A non-transitory computer-readable medium having computer-executable instructions stored thereon that, in response to execution by one or more processors of a mobile computing device, cause the mobile computing device to perform actions for managing and logging actions during an emergency care event, the actions comprising:
presenting a first modal interface element to collect information that defines at least one care guideline, and close the modal interface element after collecting the information;
in response to detecting actuation of an event-initiating interface element, presenting a non-modal interface to manage and record event information, wherein content of the at non-modal interface element is determined by the at least one care guideline; and
storing collected information in an event record in the event data store.

16. The computer-readable medium of claim 15, wherein the instructions further cause the mobile computing device to track a treatment cycle, wherein tracking a treatment cycle comprises:
starting a treatment cycle timer;
incrementing a treatment cycle counter;
presenting the treatment cycle counter, the treatment cycle timer, and a treatment cycle time remaining indicator that is based on the treatment cycle timer; and
presenting a modal alert upon detecting that the treatment cycle timer has reached a predetermined time.

17. The computer-readable medium of claim 16, wherein tracking the treatment cycle further comprises:
detecting the start of a subsequent treatment cycle;
incrementing the treatment cycle counter; and
resetting the treatment cycle timer and the treatment cycle time remaining indicator.

18. The computer-readable medium of claim 16, wherein presenting the non-modal interface includes presenting a workflow interface, and wherein presenting the workflow interface includes:
presenting an ordered list of workflow steps;
detecting actuation of a completion interface element of a first workflow step in the ordered list of workflow steps;
inserting information associated with the first workflow step into the event record; and
removing the first workflow step from the ordered list of workflow steps.

19. The computer-readable medium of claim 16, wherein presenting the non-modal interface includes presenting an order information collection interface, and wherein presenting the order information collection interface includes:
receiving a selection of an ordered item or procedure;
presenting an order information collection interface that includes a given completion interface element and an ordered completion interface element;
in response to detecting actuation of the given completion interface element, entering information in the order information collection interface into the event record; and
in response to detecting actuation of the ordered completion interface element, presenting an ordered item interface element.

20. The computer-readable medium of claim 19, wherein the actions further comprise:
detecting an ordered item interface element that has been presented for at least a threshold amount of time without being resolved; and
presenting an alert indicating that the ordered item interface element has not been resolved.
